(19) United States
(12) Patent Application Publication
(10) Pub. No.: US 2011/0251063 A1
(43) Pub. Date: Oct. 13, 2011

(54) HETEROCYCLIC DIKETONE DERIVATIVES WITH HERBICIDAL ACTION

(76) Inventors: Dschun Song, Mannheim (DE); Julia Major, Mannheim (DE); Johannes Hutzler, Waldsee (DE); Trevor William Newton, Neustadt (DE); Thomas Ehrhardt, Speyer (DE); Klaus Kreuz, Denzlingen (DE); Klaus Grossmann, Neuhofen (DE); Robert Reinhard, Limburgerhof (DE); Anja Simon, Weinheim (DE); Ricarda Niggeweg, Mannheim (DE); Bernd Sievernich, Hassloch (DE); Matthias Witschel, Bad Duerkheim (DE); William Karl Moberg, Neustadt (DE); Liliana Parra Rapado, Offenburg (DE); Tao Qu, Ludwigshafen (DE); Frank Stelzer, Mannheim (DE); Andrea Vescovi, Barcelona (ES); Thomas Seitz, Viernheim (DE)

(21) Appl. No.: 13/140,486

(22) PCT Filed: Dec. 7, 2009
(86) PCT No.: PCT/EP09/66514

§ 371 Date: Jun. 17, 2011

(30) Foreign Application Priority Data
Dec. 18, 2008 (EP) 08172182.1

(51) Int. Cl. A01N 43/40; A01P 13/00; C07D 213/65

(52) U.S. Cl. 504/103; 546/301; 504/255; 504/130

(57) ABSTRACT

Heterocyclic compounds of the formula I

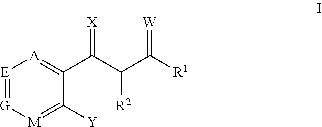

in which the variables are defined according to the description, their agriculturally suitable salts, processes and intermediates for preparing the compounds of the formula I, compositions comprising them and their use as herbicides, i.e. for controlling harmful plants, and also a method for controlling unwanted vegetation which comprises allowing a herbicidally effective amount of at least one compound of the formula I to act on plants, their seed and/or their habitat.

The present invention relates to heterocyclic compounds of the formula I

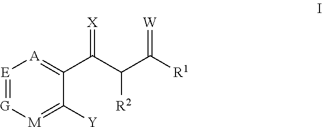

in which the variables have the following meaning:
R¹ is OR, SR or NRⁱRⁱⁱ;
   R is hydrogen, C₁-C₆-alkyl, C₁-C₄-haloalkyl, C₂-C₆-alkenyl, C₃-C₆-alkynyl, Z—C₃-C₁₀-cycloalkyl, C₁-C₆-alkoxy-C₁-C₆-alkyl, C₁-C₆-cyanoalkyl, Z-phenyl, Z—C(═O)—R^{a2}, Z—SO₂R^{a2} or tri-C₁-C₄-alkylsilyl;
      R^{a2} is C₁-C₆-alkyl, C₁-C₄-haloalkyl, Z—C₁-C₆-alkoxy, Z—C₁-C₄-halo-alkoxy or NRRⁱⁱ;
   Z is a covalent bond or C₁-C₄-alkylene;
   Rⁱ, Rⁱⁱ independently of one another are hydrogen, C₁-C₈-alkyl, C₁-C₄-haloalkyl, C₃-C₈-alkenyl, C₃-C₈-alkynyl, Z—C₃-C₈-cycloalkyl, Z—(C═O)—R^{a2}, Z—C₁-C₈-alkoxy, Z—C₁-C₈-haloalkoxy;
      Rⁱ and Rⁱⁱ together with the nitrogen atom to which they are attached may also form a 5- or 6-membered monocyclic or 9- or 10-membered bicyclic heterocycle which comprises 1, 2, 3 or 4 heteroatoms selected from the group consisting of O, N and S;
R² is phenyl, naphthyl or a 5- or 6-membered monocyclic or 9- or 10-membered bicyclic aromatic heterocycle which comprises 1, 2, 3 or 4 heteroatoms selected from the group consisting of O, N and S, where the cyclic groups are unsubstituted or substituted by 1, 2, 3, 4 or 5 groups R^{a};
   R^{a} independently of one another are Z—CN, Z—OH, Z—NO₂, Z-halogen, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, Z—C₃-C₁₀-cycloalkyl, O—Z—C₃-C₁₀-cycloalkyl, Z—C(═O)—R^{a2}, NRⁱRⁱⁱ, Z-(tri-C₁-C₄-alkyl)silyl, Z-phenyl and S(O)ₙR^{bb},
      where R^{bb} is C₁-C₈-alkyl, C₁-C₆-haloalkyl or 2-phenyl and
      n is 0, 1 or 2;
   R^{a} together with the group R^{a} attached to the adjacent carbon atom may also form a five- or six-membered saturated or partially or fully unsaturated ring which, in addition to carbon atoms, may comprise 1, 2 or 3 heteroatoms selected from the group consisting of O, N and S;
X is O, S or N—R;
Y is R¹ or NH—R;
W is O or S;
A, E, G, M are N or C—R^{c}, one or two of these groups being N;
   R^{c} is hydrogen or one of the groups mentioned for R^{a}; where in the groups R¹, R², R³ and their substituents, the carbon chains and/or the cyclic groups may be partially or fully substituted by groups R^{a}, or a N-oxide or an agriculturally suitable salt thereof.

Moreover, the invention relates to processes and intermediates for preparing the compounds of the formula I and the N-oxides thereof, the agriculturally usable salts thereof, and also to active compound combinations comprising them, to compositions comprising them and to their use as herbicides, i.e. for controlling harmful plants, and also to a method for controlling unwanted vegetation which comprises allowing a herbicidally effective amount of at least one compound of the formula I or of an agriculturally suitable salt of I to act on plants, their seed and/or their habitat.

Further embodiments of the present invention can be found in the claims, the description and the examples. It is to be understood that the features mentioned above and those still to be illustrated below of the subject matter of the invention can be applied not only in the respective given combination but also in other combinations without leaving the scope of the invention.

It is an object of the present invention to provide compounds having herbicidal action. To be provided are in particular active compounds having strong herbicidal action, in particular even at low application rates, whose compatibility with crop plants is sufficient for commercial application.

These and further objects are achieved by the compounds of the formula I defined at the outset and by their N-oxides and also their agriculturally suitable salts.

The compounds according to the invention can be prepared analogously to standard processes of organic chemistry, for example according to the following synthesis route:

The compounds of the formula I where X is O or S (═X¹) and their precursors can be present in two tautomeric forms. The invention relates to both tautomers. For the sake of clarity only, only one tautomer is generally mentioned in the description.

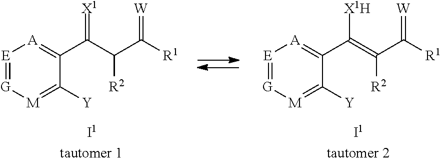

Carboxylic acids of the formula II can be reacted with compounds of the formula III to give compounds of the formula IV. In the formulae II and III, the variables have the meaning given for formula I. The group R′ is C₁-C₄-alkoxy, Y′ is O or S, Hal is a halogen atom or another suitable nucleophilic leaving group, such as alkoxy or phenoxy and SG is a protective group which decreases the reactivity of Y, such as, for example, optionally substituted benzyl.

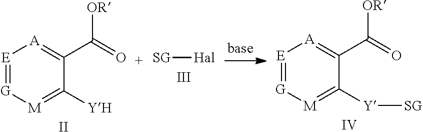

This reaction is usually carried out at temperatures of from −78° C. to 120° C., preferably from −20° C. to 50° C., in an inert organic solvent in the presence of a base, (cf. Greene's Protective Groups in Organic Synthesis, Wiley).

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, and also dimethyl sulfoxide, dimethylformamide and dimethylacetamide, particularly preferably halogenated hydrocarbons, such as methylene chloride, chloroform and chlorobenzene. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, tributylamine, diisopropylethylamine and N-methylpiperidine, pyridine, substituted pyridines, such as collidine, lutidine and 4-dimethylaminopyridine, and also bicyclic amines. Preference is given to alkali metal and alkaline earth metal hydrides, particularly preferably sodium hydride. The bases are generally employed in catalytic amounts; however, they can also be used in equimolar amounts, in excess or, if appropriate, as solvents.

The starting materials are generally reacted with one another in equimolar amounts.

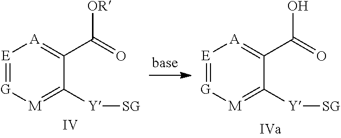

The corresponding acid of the formula IVa is released from the compounds of the formula IV. This reaction is usually carried out at temperatures of from −78° C. to 120° C., preferably from −20° C. to 50° C., in an inert organic solvent in the presence of a base (cf. Bioorganic and Medicinal Chemistry Letters (2006) Vol. 16(3), 718-721).

Suitable solvents are water, alcohols, such as methanol, ethanol, isopropanol, aliphatic hydrocarbons, such as pentane, hexane, cyclohexane and petrol ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisol and tetrahydrofuran, nitriles, such as acetonitrile and propionitrile, ketones, such as acetone, methyl ethyl ketone, diethyl ketone and tert-butyl methyl ketone, and also dimethyl sulfoxide, dimethylformamide and dimethylacetamide, particularly preferably halogenated hydrocarbons, such as methylene chloride, chloroform and chlorobenzene. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkyl magnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, tributylamine, diisopropylethylamine and N-methylpiperidine, pyridine, substituted pyridines, such as collidine, lutidine and 4-dimethylaminopyridine, and also bicyclic amines. Preference is given to alkali metal and alkaline earth metal hydroxides, particularly preferably lithium hydroxide. The bases are generally employed in catalytic amounts; however, they can also be used in equimolar amounts, in excess or, if appropriate, as solvent. The compounds of the formula IVa are activated by introducing a leaving group L¹. Suitable leaving groups L¹ are, in general, groups which increase the electrophilicity of the carbonyl group, for example O-alkyl, O-aryl, halides, activated esters or aldehydes (such as, for example, Weinreb amide), in particular pentafluorophenoxy.

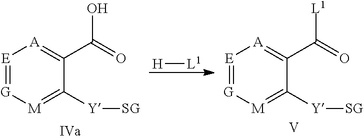

This reaction is usually carried out at temperatures of from −78° C. to 120° C., preferably from −20° C. to 50° C., in an inert organic solvent in the presence of a base, such as, for example, triethylamine (cf. J. Agric. and Food Chem. 1994, 42(4), 1019-1025), a catalyst, such as, for example, dicyclohexylcarbodiimide (cf. Egyptian Journal of Chemistry 1994, 37(3), 273-282) or other known coupling agents.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, and also dimethyl sulfoxide, dimethylformamide and dimethylacetamide, particularly preferably methylene chloride and toluene. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, tributylamine, diisopropylethylamine and N-methylpiperidine, pyridine, substituted pyridines, such as collidine, lutidine and 4-dimethylaminopyridine, and also bicyclic amines. Particular preference is given to alkali metal and alkaline earth metal carbonates, such as lithium carbonate, potassium carbonate, calcium carbonate, cesium carbonate and rubidium carbonate. The bases are generally employed in catalytic amounts; however, they can also be used in equimolar amounts, in excess or, if appropriate, as solvents.

The starting materials are generally reacted with one another in equimolar amounts.

Suitable agents H-L¹ are alcohols, optionally subst. phenols, N,O-dialkyl-hydroxylamine, in particular pentafluorophenol or N,O-dimethylhydroxylamine.

The compounds of the formula V are reacted with acetic acid derivatives of the formula VI to give the compounds of the formula VII.

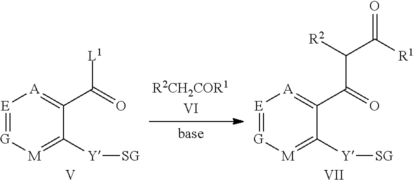

This reaction is usually carried out at temperatures of from −78° C. to 120° C., preferably from −20° C. to 50° C., in an inert organic solvent in the presence of a base or a Lewis acid or a catalyst [cf. Bioorganic & Medicinal Chemistry (2004) Vol. 12(6), 1357-1366].

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chloro-benzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, and also dimethyl sulfoxide, dimethylformamide and dimethylacetamide, particularly preferably acetonitrile and dimethylformamide. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate, calcium carbonate, cesium carbonate and rubidium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, tributylamine, diisopropylethylamine and N-methylpiperidine, pyridine, substituted pyridines, such as collidine, lutidine and 4-dimethylaminopyridine, and also bicyclic amines. Preference is given to alkali metal and alkaline earth metal alkoxides, particularly preferably potassium tert-butoxide.

The bases are generally employed in catalytic amounts; however, they can also be used in equimolar amounts, in excess or, if appropriate, as solvents.

The starting materials are generally reacted with one another in equimolar amounts.

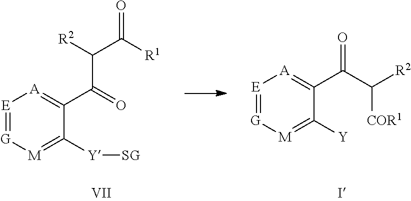

The compounds of the formula I′ can be released from the compounds of the formula VII by removal of the protective group. The reaction conditions depend on the nature of the protective group SG; the removal of an optionally substituted benzyl group can be achieved, for example, using trifluoroacetic acid at temperatures of from −78° C. to 100° C., preferably from −20° C. to 50° C., in an inert organic solvent [cf. Greene's Protective Groups in Organic Synthesis, Wiley].

The introduction of the group X or Y different from oxygen into the compounds of the formula I′ or precursors thereof is carried out according to generally known methods.

Tautomer 2 of the compounds of the formula I can be alkylated, acylated or sulfonated by reaction with, respectively, an alkylating, acylating or sulfonating agent of formula VIII according to generally known methods.

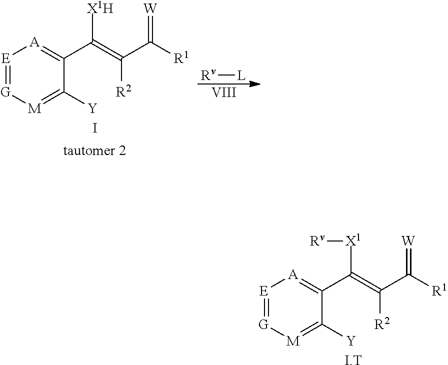

In the formula VIII, R^{v} is C₁-C₈-alkyl, C₁-C₈-alkylcarbonyl, C₁-C₈-alkylsulfonyl, C₁-C₈-arylcarbonyl, C₁-C₈-arylsulfonyl, wherein the alkyl and aryl groups may be partially or fully substituted by groups R^{a}, and L is a nucleophilic leaving group, for example halogen, alkoxy, alkylcarbonyloxy or arylcarbonyloxy. Useful acylating or sulfonating agents include, in particular, acetyl chloride, pivaloyl chloride, tosyl chloride or methyl-sulfonyl chloride.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, the purification can also be carried out by recrystallization or digestion.

If individual compounds I cannot be obtained by the routes described above, they can be prepared by derivatization of other compounds I.

If the synthesis yields mixtures of isomers, a separation is, generally however not necessarily required since in some cases the individual isomers can be interconverted during work-up for use or during application (for example under the action of light, acids or bases). Such conversions may also take place after application, for example in the case of the treatment of plants in the treated plant or in the harmful plant to be controlled.

The organic moieties mentioned for the substituents of the compounds according to the invention are collective terms for individual enumerations of the individual group members. All hydrocarbon chains, such as alkyl, haloalkyl, alkenyl, alkynyl, and the alkyl moieties and alkenyl moieties in alkoxy, haloalkoxy, alkylamino, dialkylamino, N-alkylsulfonylamino, alkenyloxy, alkynyloxy, alkoxyamino, alkylaminosulfonylamino, dialkylaminosulfonylamino, alkenylamino, alkynylamino, N-(alkenyl)-N-(alkyl)amino, N-(alkynyl)-N-(alkyl)amino, N-(alkoxy)-N-(alkyl)amino, N-(alkenyl)-N-(alkoxy)amino or N-(alkynyl)-N-(alkoxy)amino can be straight-chain or branched.

The prefix Cₙ-Cₘ- indicates the respective number of carbons of the hydrocarbon unit. Unless indicated otherwise, halogenated substituents preferably carry one to five identical or different halogen atoms, in particular fluorine atoms or chlorine atoms.

The meaning halogen denotes in each case fluorine, chlorine, bromine or iodine.

Examples of other meanings are:

alkyl and the alkyl moieties for example in alkoxy, alkylamino, dialkylamino, N-alkyl-sulfonylamino, alkylaminosulfonylamino, dialkylaminosulfonylamino, N-(alkenyl)-N-(alkyl)amino, N-(alkynyl)-N-(alkyl)amino, N-(alkoxy)-N-(alkyl)amino: saturated straight-chain or branched hydrocarbon radicals having one or more carbon atoms, for example 1 or 2, 1 to 4 or 1 to 6 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethyl-butyl, 2,3-dimethylbutyl, 3,3-dimethylbutyl, 1-ethylbutyl, 2-ethylbutyl, 1,1,2-trimethyl-propyl, 1,2,2-trimethylpropyl, 1-ethyl-1-methylpropyl, 1-ethyl-2-methylpropyl. In one embodiment according to the invention, alkyl denotes small alkyl groups, such as C₁-C₄-alkyl. In another embodiment according to the invention, alkyl denotes relatively large alkyl groups, such as C₅-C₆-alkyl.

Haloalkyl: an alkyl radical as mentioned above, some or all of whose hydrogen atoms are substituted by halogen atoms, such as fluorine, chlorine, bromine and/or iodine, for example chloromethyl, dichloromethyl, trichloromethyl, fluoromethyl, difluoro-methyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoro-ethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl and nonafluorobutyl.

Cycloalkyl and the cycloalkyl moieties for example in cycloalkoxy or cycloalkylcarbonyl: monocyclic saturated hydrocarbon groups having three or more carbon atoms, for example 3 to 6 carbon ring members, such as cyclopropyl, cyclobutyl, cyclopentyl and cyclohexyl.

Alkenyl and the alkenyl moieties for example in alkenylamino, alkenyloxy, N-(alkenyl)-N-(alkyl)amino, N-(alkenyl)-N-(alkoxy)amino: monounsaturated straight-chain or branched hydrocarbon radicals having two or more carbon atoms, for example 2 to 4, 2 to 6 or 3 to 6 carbon atoms, and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl.

Cycloalkenyl: monocyclic monounsaturated hydrocarbon groups having 3 to 6, preferably 5 or 6, carbon ring members, such as cyclopenten-1-yl, cyclopenten-3-yl, cyclohexen-1-yl, cyclohexen-3-yl, cyclohexen-4-yl.

Alkynyl and the alkynyl moieties for example in alkynyloxy, alkynylamino, N-(alkynyl)-N-(alkyl)amino or N-(alkynyl)-N-(alkoxy)amino: straight-chain or branched hydrocarbon groups having two or more carbon atoms, for example 2 to 4, 2 to 6 or 3 to 6 carbon atoms, and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl.

Alkoxy: alkyl as defined above which is attached via an oxygen atom, for example methoxy, ethoxy, n-propoxy, 1-methylethoxy, butoxy, 1-methylpropoxy, 2-methyl-propoxy or 1,1-dimethylethoxy, pentoxy, 1-methylbutoxy, 2-methylbutoxy, 3-methyl-butoxy, 1,1-dimethylpropoxy, 1,2-dimethylpropoxy, 2,2-dimethylpropoxy, 1-ethyl-propoxy, hexoxy, 1-methylpentoxy, 2-methylpentoxy, 3-methylpentoxy, 4-methyl-pentoxy, 1,1-dimethylbutoxy, 1,2-dimethylbutoxy, 1,3-dimethylbutoxy, 2,2-dimethyl-butoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

A 5- or 6-membered heterocycle: a cyclic group which has 5 or 6 ring atoms, 1, 2, 3 or 4 ring atoms being heteroatoms selected from the group consisting of O, S and N, where the cyclic group is saturated, partially unsaturated or aromatic. Examples of heterocyclic groups are.

The compounds of the formula I may, depending on the substitution pattern, comprise one or more further centers of chirality. Accordingly, the compounds according to the invention can be present as pure enantiomers or diastereomers or as enantiomer or diastereomer mixtures. The invention provides both the pure enantiomers or diastereomers and their mixtures.

The compounds of the formula I may also be present in the form of the N-oxides and/or of their agriculturally useful salts, the type of salt generally not being important. Suitable salts are generally the salts of those cations or the acid addition salts of those acids whose cations and anions, respectively, have no adverse effect on the herbicidal activity of the compounds I.

Suitable cations are in particular ions of the alkali metals, preferably lithium, sodium or potassium, of the alkaline earth metals, preferably calcium or magnesium, and of the transition metals, preferably manganese, copper, zinc or iron. Another cation that may be used is ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropyl-ammonium, tetramethylammonium, tetrabutylammonium, 2-(2-hydroxyeth-1-oxy)eth-1-ylammonium, di(2-hydroxyeth-1-yl)ammonium, trimethylbenzylammonium. Another suitable ammonium cation is the pyridine nitrogen atom of the formula I quaternized by alkylation or arylation. Also suitable are phosphonium ions, sulfonium ions, preferably tri(C₁-C₄-alkyl)sulfonium, or sulfoxonium ions, preferably tri(C₁-C₄-alkyl)sulfoxonium.

Anions of suitable acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate, butyrate or trifluoroacetate.

With respect to the variables, the particularly preferred embodiments of the intermediates correspond to those of the groups of the formula I.

In a particular embodiment, the variables of the compounds of the formula I have the following meanings, these meanings, both on their own and in combination with one another, being particular embodiments of the compounds of the formula I:

In one preferred embodiment of the compounds of the formula I, a group consists of A, E, G and M is N.

In one preferred embodiment of the compounds of the formula I, A is N and E, G and M are C—R^{a}. These compounds correspond to the formula I.1

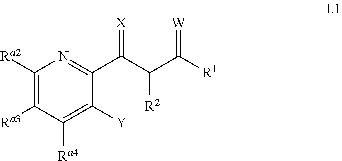

in which W is preferably O and the groups R^{a2}, R^{a3} and R^{a4} each correspond to a group R^{a} and preferably have the following meanings:
R^{a2} is H, OH, CN, halogen, alkyl, alkoxy, haloalkyl, in particular H, Br, OH and OCH₃;
R^{a3} is H, OH, CN, halogen, alkyl, alkoxy, haloalkyl, in particular H;
R^{a4} is H, OH, CN, halogen, alkyl, alkoxy, haloalkyl, in particular H.

In a further preferred embodiment of the compounds of the formula I, A and M are each N and E and G are each C—R^{a}. These compounds correspond to the formula I.2,

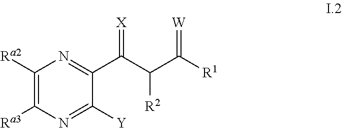

where W is preferably O and the groups R^{a2} and R^{a3} each correspond to a group R^{a} and preferably have the following meanings:
R^{a2}: H, OH, CN, halogen, alkyl, alkoxy, haloalkyl, in particular H, Br, OH and OCH₃;
R^{a3}: H, OH, CN, halogen, alkyl, alkoxy, haloalkyl, in particular H.

In a first preferred embodiment of the invention, R¹ is alkoxy, in particular methoxy.

In a further preferred embodiment, R is H, C₁-C₆-alkyl, such as CH₃, C₂H₅, n-C₃H₇, CH(CH₃)₂, n-C₃H₉, or C(CH₃)₃; C₃-C₆-cycloalkyl-C₁-C₄-alkyl, such as cyclopropylmethyl, C₃-C₆-alkenyl, such as CH₂CH═CH₂, CH₂C(CH₃)═CH₂, CH₂CH₂H═CH₂, CH₂CH₂C(CH₃)—CH₂, CH₂CH₂CH₂CH═CH₂, CH₂CH₂CH₂C(CH₃)═CH₂, or optionally subst. phenyl, such as C₆-C₅, 4-CH₃-C₆, 4-F—C₆H₄ or S(O)ₙ—R^{N}, where R^{N} is C₁-C₅-haloalkyl, such as CH₂CF₃, CH₂CHF₂.

In a further preferred aspect, R² is phenyl which is unsubstituted or partially or fully substituted by groups R^{a}. Particular preference is given to compounds in which a group R^{a} is located in the ortho-position. Such compounds of the formula I are described by the formula I.A:

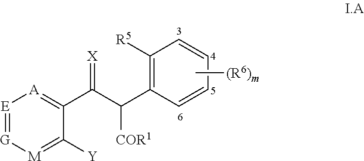

In formula I.A, the index m is an integer from zero to four, preferably 0, 1 or 2, in particular 0 or 1. R⁵ and R⁶ are groups R^{a} as defined at the outset, preferably halogen, NO₂, C₁-C₄-alkyl, C₁-C₂-haloalkyl, C₁-C₄-alkoxy and C₁-C₂-haloalkoxy. One group R⁶ is preferably located in position 5.

In a preferred embodiment, X is O.

In a further embodiment, X is S.

In a further embodiment, X is NR.

In a preferred embodiment, Y is OH.

In a further embodiment, Y is SH.

In a further embodiment, Y is NHR.

In a preferred embodiment, W is O.

In a further embodiment, W is S.

A further preferred embodiment comprises the compounds of the formula I which are derived from tautomer 2; they correspond to the formula I.T.

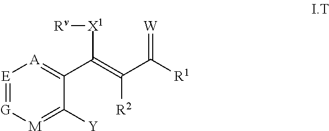

The particularly preferred embodiments of the compounds of the formula I.T with regard to the variables correspond to those of the groups of formula I.

R^{v} is preferably C₁-C₈-alkylcarbonyl, C₁-C₈-alkylsulfonyl, C₁-C₈-arylcarbonyl, or C₁-C₈-arylsulfonyl, wherein the alkyl and aryl groups may be partially or fully substituted by groups R^{a}. Aryl is preferably phenyl. Particular preference is given to C₁-C₈-alkyl-carbonyl and C₁-C₈-alkylsulfonyl, for example acetyl, pivaloyl, tosyl or methylsulfonyl.

A further embodiment relates to the N-oxides of the compounds of the formula I. A further embodiment relates to salts of the compounds of the formula I, in particular those which are obtainable by quaternization of the pyridine nitrogen atom, which may preferably take place by alkylation or arylation of the compounds of the formula I. Preferred salts of the compounds are thus the N-alkyl salts, in particular the N-methyl salts, and the N-phenyl salts.

In particular with a view to their use, preference is given to the compounds of the formula I compiled in the tables below, which compounds correspond to the formula I.1A. The groups mentioned for a substituent in the tables are furthermore per se, independently of the combination in which they are mentioned, a particularly preferred aspect of the substituent in question.

Table 1
Compounds of the formula I in which X is O, Y is OH and R¹ is CH₃ and the combination of R⁵ and (R⁶)ₘ for a compound corresponds in each case to one row of table A

Table 2
Compounds of the formula I in which X is O, Y is SH and R¹ is CH₃ and the combination of R⁵ and (R⁶)ₘ for a compound corresponds in each case to one row of table A

Table 3
Compounds of the formula I in which X is O, Y is NH₂ and R¹ is CH₃ and the combination of R⁵ and (R⁶)ₘ for a compound corresponds in each case to one row of table A

Table 4
Compounds of the formula I in which X is O, Y is NH and R¹ is CH₃ and the combination of R⁵ and (R⁶)ₘ for a compound corresponds in each case to one row of table A

Table 5
Compounds of the formula I in which X is O, Y is NH—C₂H₅ and R¹ is CH₃ and the combination of R⁵ and (R⁶)ₘ for a compound corresponds in each case to one row of table A

Table 6
Compounds of the formula I in which X is O, Y is NH-n-C₃H₇ and R¹ is CH₃ and the combination of R⁵ and (R⁶)ₘ for a compound corresponds in each case to one row of table A

Table 7
Compounds of the formula I in which X is O, Y is NH—CH(CH₃)₂ and R¹ is CH₃ and the combination of R⁵ and (R⁶)ₘ for a compound corresponds in each case to one row of table A

Table 8
Compounds of the formula I in which X is O, Y is NH-n-C₄H₉ and R¹ is CH₃ and the combination of R⁵ and (R⁶)ₘ for a compound corresponds in each case to one row of table A

Table 9
Compounds of the formula I in which X is O, Y is NH—CH₂CH═CH₂ and R¹ is CH₃ and the combination of R⁵ and (R⁶)ₘ for a compound corresponds in each case to one row of table A

Table 10
Compounds of the formula I in which X is O, Y is NH—CH₂C═CH and R¹ is CH₃ and the combination of R⁵ and (R⁶)ₘ for a compound corresponds in each case to one row of table A

Table 11
Compounds of the formula I in which X is O, Y is NH—CH₂CN and R¹ is CH₃ and the combination of R⁵ and (R⁶)ₘ for a compound corresponds in each case to one row of table A

Table 12
Compounds of the formula I in which X is O, Y is NH—CH₂C₆H5 and R¹ is CH₃ and the combination of R⁵ and (R⁶)ₘ for a compound corresponds in each case to one row of table A

Table 13
Compounds of the formula I in which X is S, Y is SH and R¹ is CH₃ and the combination of R⁵ and (R⁶)ₘ for a compound corresponds in each case to one row of table A

Table 14
Compounds of the formula I in which X is S, Y is OH and R¹ is CH₃ and the combination of R⁵ and (R⁶)ₘ for a compound corresponds in each case to one row of table A

Table 15
Compounds of the formula I in which X is S, Y is NH₂ and R¹ is CH₃ and the combination of R⁵ and (R⁶)ₘ for a compound corresponds in each case to one row of table A

Table 16
Compounds of the formula I in which X is S, Y is NH—CH₃ and R¹ is CH₃ and the combination of R⁵ and (R⁶)ₘ for a compound corresponds in each case to one row of table A

Table 17
Compounds of the formula I in which X is S, Y is NH—C₂H₅ and R¹ is CH₃ and the combination of R⁵ and (R⁸)ₘ for a compound corresponds in each case to one row of table A

Table 18
Compounds of the formula I in which X is S, Y is NH-n-C₃H₇ and R¹ is CH₃ and the combination of R⁵ and (R⁶)ₘ for a compound corresponds in each case to one row of table A

Table 19
Compounds of the formula I in which X is S, Y is NH—CH(CH₃)2 and R¹ is CH₃ and the combination of R⁵ and (R⁶)ₘ for a compound corresponds in each case to one row of table A

Table 20
Compounds of the formula I in which X is S, Y is NH-n-C₄H₉ and R¹ is CH₃ and the combination of R⁵ and (R⁶)ₘ for a compound corresponds in each case to one row of table A

Table 21
Compounds of the formula I in which X is S, Y is NH—CH₂CH═CH₂ and R¹ is CH₃ and the combination of R⁵ and (R⁶)ₘ for a compound corresponds in each case to one row of table A

Table 22
Compounds of the formula I in which X is S, Y is NH—CH₂C═CH and R¹ is CH₃ and the combination of R⁵ and (R⁶)ₘ for a compound corresponds in each case to one row of table A

Table 23
Compounds of the formula I in which X is S, Y is NH—CH₂CN and R¹ is CH₃ and the combination of R⁵ and (R⁶)ₘ for a compound corresponds in each case to one row of table A

Table 24
Compounds of the formula I in which X is S, Y is NH—CH₂C₆H₅ and R¹ is CH₃ and the combination of R⁵ and (R⁶)ₘ for a compound corresponds in each case to one row of table A

TABLE A
Compounds of the formula I which correspond to the formula I.1A
I.1A

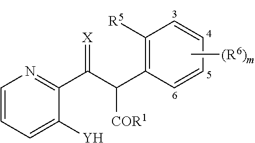

| No. | R⁵ | (R⁶)ₘ |
|---|---|---|
| A-1 | Br | — |
| A-2 | Cl | — |
| A-3 | F | — |
| A-4 | NO₂ | — |
| A-5 | CN | — |
| A-6 | CH₃ | — |
| A-7 | OCH₃ | — |
| A-8 | CHF₂ | — |
| A-9 | CF₃ | — |
| A-10 | OCHF₂ | — |
| A-11 | OCF₃ | — |
| A-12 | Cl | 4-Cl |
| A-13 | F | 4-Cl |
| A-14 | NO₂ | 4-Cl |
| A-15 | CH₃ | 4-Cl |
| A-16 | OCH₃ | 4-Cl |
| A-17 | CHF₂ | 4-Cl |
| A-18 | CF₃ | 4-Cl |
| A-19 | OCHF₂ | 4-Cl |
| A-20 | OCF₃ | 4-Cl |
| A-21 | Cl | 3-F |
| A-22 | F | 3-F |
| A-23 | NO₂ | 3-F |
| A-24 | CH₃ | 3-F |
| A-25 | OCH₃ | 3-F |
| A-26 | CHF₂ | 3-F |
| A-27 | CF₃ | 3-F |
| A-28 | OCHF₂ | 3-F |
| A-29 | OCF₃ | 3-F |
| A-30 | Cl | 4-F |
| A-31 | F | 4-F |
| A-32 | NO₂ | 4-F |
| A-33 | CH₃ | 4-F |
| A-34 | OCH₃ | 4-F |
| A-35 | CHF₂ | 4-F |
| A-36 | CF₃ | 4-F |
| A-37 | OCHF₂ | 4-F |
| A-38 | OCF₃ | 4-F |
| A-39 | Cl | 5-F |
| A-40 | F | 5-F |
| A-41 | NO₂ | 5-F |
| A-42 | CH₃ | 5-F |
| A-43 | OCH₃ | 5-F |
| A-44 | CHF₂ | 5-F |
| A-45 | CF₃ | 5-F |
| A-46 | OCHF₂ | 5-F |
| A-47 | OCF₃ | 5-F |
| A-48 | Cl | 6-F |
| A-49 | F | 6-F |
| A-50 | NO₂ | 6-F |
| A-51 | CH₃ | 6-F |
| A-52 | OCH₃ | 6-F |
| A-53 | CHF₂ | 6-F |
| A-54 | CF₃ | 6-F |
| A-55 | OCHF₂ | 6-F |
| A-56 | OCF₃ | 6-F |
| A-57 | Cl | 4-CF₃ |
| A-58 | F | 4-CF₃ |
| A-59 | NO₂ | 4-CF₃ |
| A-60 | CH₃ | 4-CF₃ |
| A-61 | OCH₃ | 4-CF₃ |
| A-62 | CHF₂ | 4-CF₃ |
| A-63 | CF₃ | 4-CF₃ |
| A-64 | OCHF₂ | 4-CF₃ |
| A-65 | OCF₃ | 4-CF₃ |
| A-66 | Cl | 5-CF₃ |
| A-67 | F | 5-CF₃ |
| A-68 | NO₂ | 5-CF₃ |
| A-69 | CH₃ | 5-CF₃ |
| A-70 | OCH₃ | 5-CF₃ |
| A-71 | CHF₂ | 5-CF₃ |
| A-72 | CF₃ | 5-CF₃ |
| A-73 | OCHF₂ | 5-CF₃ |
| A-74 | OCF₃ | 5-CF₃ |
| A-75 | Cl | 3,6-F₂ |
| A-76 | F | 3,6-F₂ |
| A-77 | NO₂ | 3,6-F₂ |
| A-78 | CH₃ | 3,6-F₂ |
| A-79 | OCH₃ | 3,6-F₂ |
| A-80 | CHF₂ | 3,6-F₂ |
| A-81 | CF₃ | 3,6-F₂ |
| A-82 | OCHF₂ | 3,6-F₂ |
| A-83 | OCF₃ | 3,6-F₂ |
| A-84 | Br | 4-OCF₃, 6-Cl |
| A-85 | Cl | 4-OCF₃, 6-Cl |
| A-86 | F | 4-OCF₃, 6-Cl |
| A-87 | NO₂ | 4-OCF₃, 6-Cl |
| A-88 | CN | 4-OCF₃, 6-Cl |
| A-89 | CH₃ | 4-OCF₃, 6-Cl |
| A-90 | OCH₃ | 4-OCF₃, 6-Cl |
| A-91 | CHF₂ | 4-OCF₃, 6-Cl |
| A-92 | CF₃ | 4-OCF₃, 6-Cl |
| A-93 | OCHF₂ | 4-OCF₃, 6-Cl |
| A-94 | OCF₃ | 4-OCF₃, 6-Cl |

The compounds I and their agriculturally useful salts are suitable, both as isomer mixtures and in the form of the pure isomers, as herbicides. They are suitable as such or as an appropriately formulated composition. The herbicidal compositions comprising the compound I, in particular the preferred aspects thereof, control vegetation on non-crop areas very efficiently, especially at high rates of application. They act against broad-leaved weeds and weed grasses in crops such as wheat, rice, corn, soybeans and cotton without causing any significant damage to the crop plants. This effect is mainly observed at low rates of application.

Depending on the application method in question, the compounds I, in particular the preferred aspects thereof, or compositions comprising them can additionally be employed in a further number of crop plants for eliminating unwanted plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica raps var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera, Zea mays.

The term “crop plants” also includes plants which have been modified by breeding, mutagenesis or genetic engineering. Genetically modified plants are plants whose genetic material has been modified in a manner which does not occur under natural conditions by crossing, mutations or natural recombination (i.e. reassembly of the genetic information). Here, in general, one or more genes are integrated into the genetic material of the plant to improve the properties of the plant.

Accordingly, the term “crop plants” also includes plants which, by breeding and genetic engineering, have acquired tolerance to certain classes of herbicides, such as ⁻hydroxyphenylpyruvate dioxygenase (HPPD) inhibitors, acetolactate synthase (ALS) inhibitors, such as, for example, sulfonylureas (EP-A 257 993, U.S. Pat. No. 5,013,659) or imidazolinones (see, for example, U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), enolpyruvylshikimate 3-phosphate synthase (EPSPS) inhibitors, such as, for example, glyphosate (see, for example, WO 92/00377), glutamine synthetase (GS) inhibitors, such as, for example, glufosinate (see, for example, EP-A 242 236, EP-A 242 246), or oxynil herbicides (see, for example, U.S. Pat. No. 5,559,024).

Numerous crop plants, for example Clearfield oilseed rape, tolerant to imidazolinones, for example imazamox, have been generated with the aid of classic breeding methods (mutagenesis). Crop plants such as soybeans, cotton, corn, beet and oilseed rape, resistant to glyphosate or glufosinate, which are available under the tradenames RoundupReady® (glyphosate) and Liberty Link® (glufosinate) have been generated with the aid of genetic engineering methods.

Accordingly, the term “crop plants” also includes plants which, with the aid of genetic engineering, produce one or more toxins, for example those of the bacterial strain Bacillus ssp. Toxins which are produced by such genetically modified plants include, for example, insecticidal proteins of Bacillus spp., in particular B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), for example VIP1, VIP2, VIP3, or VIP3A; insecticidal proteins of nematode-colonizing bacteria, for example Photorhabdus spp. or Xenorhabdus spp.; toxins of animal organisms, for example wasp, spider or scorpion toxins; fungal toxins, for example from Streptomycetes; plant lectins, for example from peas or barley; agglutinins; proteinase inhibitors, for example trypsin inhibitors, serine protease inhibitors, patatin, cystatin or papain inhibitors, ribosome-inactivating proteins (RIPs), for example ricin, corn-RIP, abrin, luffin, saporin or bryodin; steroid-metabolizing enzymes, for example 3-hydroxysteroid oxidase, ecdysteroid-IDP glycosyl transferase, cholesterol oxidase, ecdysone inhibitors, or HMG-CoA reductase; ion channel blockers, for example inhibitors of sodium channels or calcium channels; juvenile hormone esterase; receptors of the diuretic hormone (helicokinin receptors); stilbene synthase, bibenzyl synthase, chitinases and glucanases. In the plants, these toxins may also be produced as pretoxins, hybrid proteins or truncated or otherwise modified proteins. Hybrid proteins are characterized by a novel combination of different protein domains (see, for example, WO 2002/015701). Further examples of such toxins or genetically modified plants which produce these toxins are disclosed in EP-A 374 753, WO 93/007278, WO 95/34656, EP-A 427 529, EP-A 451 878, WO 03/018810 and WO 03/052073. The methods for producing these genetically modified plants are known to the person skilled in the art and disclosed, for example, in the publications mentioned above. Numerous of the toxins mentioned above bestow, upon the plants by which they are produced, tolerance to pests from all taxonomic classes of arthropods, in particular to beetles (Coeleropta), dipterans (Diptera) and butterflies (Lepidoptera) and to nematodes (Nematoda).

Genetically modified plants which produce one or more genes coding for insecticidal toxins are described, for example, in the publications mentioned above, and some of them are commercially available, such as, for example, YieldGard® (corn varieties producing the toxin Cry1Ab), YieldGard® Plus (corn varieties which produce the toxins Cry1Ab and Cry3Bb1), Starlink® (corn varieties which produce the toxin Cry9c), Herculex® RW (corn varieties which produce the toxins Cry34Ab1, Cry35Ab1 and the enzyme phosphinothricin-N-acetyltransferase [PAT]); NuCOTN® 33B (cotton varieties which produce the toxin Cry1Ac), Bollgard® I (cotton varieties which produce the toxin Cry1Ac), Bolgard® II (cotton varieties which produce the toxins Cry1Ac and Cry2Ab2); VIPCOT® (cotton varieties which produce a VIP toxin); NewLeaf® (potato varieties which produce the toxin Cry3A); Bt-Xtra®, NatureGard®, KnockOut®, BiteGard®, Protecta®, Bt11 (for example Agrisure® CB) and Bt176 from Syngenta Seeds SAS, France (corn varieties which produce the toxin Cry1Ab and the PAT enyzme), MIR⁶⁰⁴ from Syngenta Seeds SAS, France (corn varieties which produce a modified version of the toxin Cry3A, see WO 03/018810), MON 863 from Monsanto Europe S.A., Belgium (corn varieties which produce the toxin Cry3Bb1), IPC 531 from Monsanto Europe S.A., Belgium (cotton varieties which produce a modified version of the toxin Cry1Ac) and 1507 from Pioneer Overseas Corporation, Belgium (corn varieties which produce the toxin Cry1F and the PAT enzyme).

Accordingly, the term “crop plants” also includes plants which, with the aid of genetic engineering, produce one or more proteins which are more robust or have increased resistance to bacterial, viral or fungal pathogens, such as, for example, pathogenesis-related proteins (PR proteins, see EP-A 392 225), resistance proteins (for example potato varieties producing two resistance genes against Phytophthora infestans from the wild Mexican potato Solarium bulbocastanum) or T4 lysozyme (for example potato cultivars which, by producing this protein, are resistant to bacteria such as Erwinia amylvora).

Accordingly, the term “crop plants” also includes plants whose productivity has been improved with the aid of genetic engineering methods, for example by enhancing the potential yield (for example biomass, grain yield, starch, oil or protein content), tolerance to drought, salt or other limiting environmental factors or resistance to pests and fungal, bacterial and viral pathogens.

The term “crop plants” also includes plants whose ingredients have been modified with the aid of genetic engineering methods in particular for improving human or animal diet, for example by oil plants producing health-promoting long-chain omega 3 fatty acids or monounsaturated omega 9 fatty acids (for example Nexera® oilseed rape).

The term “crop plants” also includes plants which have been modified with the aid of genetic engineering methods for improving the production of raw materials, for example by increasing the amylopectin content of potatoes (Amflora® potato).

Furthermore, it has been found that the compounds of the formula I are also suitable for the defoliation and/or desiccation of plant parts, for which crop plants such as cotton, potato, oilseed rape, sunflower, soybean or field beans, in particular cotton, are suitable. In this regard, there have been found compositions for the desiccation and/or defoliation of plants, processes for preparing these compositions and methods for desiccating and/or defoliating plants using the compounds of the formula I.

As desiccants, the compounds of the formula I are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pomaceous fruit, stone fruit and nuts. The same mechanism, i.e. the promotion of the development of abscission tissue between fruit part or leaf part and shoot part of the plants is also essential for the readily controllable defoliation of useful plants, in particular cotton.

Moreover, a shortening of the time interval in which the individual cotton plants mature leads to an increased fiber quality after harvesting.

The compounds I, or the herbicidal compositions comprising the compounds I, can be used, for example, in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading, watering or treatment of the seed or mixing with the seed. The use forms depend on the intended purpose; in each case, they should ensure the finest possible distribution of the active ingredients according to the invention.

The herbicidal compositions comprise a herbicidally effective amount of at least one compound of the formula I or an agriculturally useful salt of I, and auxiliaries which are customary for the formulation of crop protection agents.

Examples of auxiliaries customary for the formulation of crop protection agents are inert auxiliaries, solid carriers, surfactants (such as dispersants, protective colloids, emulsifiers, wetting agents and tackifiers), organic and inorganic thickeners, bactericides, antifreeze agents, antifoams, if appropriate colorants and, for seed formulations, adhesives.

Examples of thickeners (i.e. compounds which impart to the formulation modified flow properties, i.e. high viscosity in the state of rest and low viscosity in motion) are polysaccharides, such as xanthan gum (Kelzan® from Kelco), Rhodopol® 23 (Rhone Poulenc) or Veegum® (from R. T. Vanderbilt), and also organic and inorganic sheet minerals, such as Attaclay® (from Engelhardt).

Examples of antifoams are silicone emulsions (such as, for example, Silikon® SRE, Wacker or Rhodorsil® from Rhodia), long-chain alcohols, fatty acids, salts of fatty acids, organofluorine compounds and mixtures thereof.

Bactericides can be added for stabilizing the aqueous herbicidal formulation. Examples of bactericides are bactericides based on diclorophen and benzyl alcohol hemiformal (Proxel® from ICI or Acticide® RS from Thor Chemie and Kathon® MK from Rohm & Haas), and also isothiazolinone derivates, such as alkylisothiazolinones and benzisothiazolinones (Acticide MBS from Thor Chemie).

Examples of antifreeze agents are ethylene glycol, propylene glycol, urea or glycerol.

Examples of colorants are both sparingly water-soluble pigments and water-soluble dyes. Examples which may be mentioned are the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohol and tylose.

Suitable inert auxiliaries are, for example, the following:

mineral oil fractions of medium to high boiling point, such as kerosene and diesel oil, furthermore coal tar oils and oils of vegetable or animal origin, aliphatic, cyclic and aromatic hydrocarbons, for example paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone or strongly polar solvents, for example amines such as N-methylpyrrolidone, and water.

Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

Suitable surfactants (adjuvants, wetting agents, tackifiers, dispersants and also emulsifiers) are the alkali metal salts, alkaline earth metal salts and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF SE), and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkyiphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denatured proteins, polysaccharides (e.g. methylcellulose), hydrophobically modified starches, polyvinyl alcohol (Mowiol types Clariant), polycarboxylates (BASF SE, Sokalan types), polyalkoxylates, polyvinylamine (BASF SE, Lupamine types), polyethyleneimine (BASF SE, Lupasol types), polyvinylpyrrolidone and copolymers thereof.

Powders, materials for broadcasting and dusts can be prepared by mixing or grinding the active ingredients together with a solid carrier.

Granules, for example coated granules, impregnated granules and homogeneous granules, can be prepared by binding the active ingredients to solid carriers.

Aqueous use forms can be prepared from emulsion concentrates, suspensions, pastes, wettable powders or water-dispersible granules by adding water. To prepare emulsions, pastes or oil dispersions, the compounds of the formula I or la, either as such or dissolved in an oil or solvent, can be homogenized in water by means of a wetting agent, tackifier, dispersant or emulsifier. Alternatively, it is also possible to prepare concentrates comprising active substance, wetting agent, tackifier, dispersant or emulsifier and, if desired, solvent or oil, which are suitable for dilution with water.

The concentrations of the compounds of the formula I in the ready-to-use preparations can be varied within wide ranges. In general, the formulations comprise from 0.001 to 98% by weight, preferably 0.01 to 95% by weight of at least one active compound. The active compounds are employed in a purity of from 90% to 100%, preferably 95% to 100% (according to NMR spectrum).

The compounds I of the invention can for example be formulated as follows:

1. Products for Dilution with Water

A Water-Soluble Concentrates

10 parts by weight of active compound are dissolved in 90 parts by weight of water or a water-soluble solvent. As an alternative, wetters or other adjuvants are added. The active compound dissolves upon dilution with water. This gives a formulation with an active compound content of 10% by weight.

B Dispersible Concentrates

20 parts by weight of active compound are dissolved in 70 parts by weight of cyclohexanone with addition of 10 parts by weight of a dispersant, for example polyvinylpyrrolidone. Dilution with water gives a dispersion. The active compound content is 20% by weight.

C Emulsifiable Concentrates

15 parts by weight of active compound are dissolved in 75 parts by weight of an organic solvent (e.g. alkylaromatics) with addition of calcium dodecylbenzenesulfonate and castor oil ethoxylate (in each case 5 parts by weight). Dilution with water gives an emulsion. The formulation has an active compound content of 15% by weight.

D Emulsions

25 parts by weight of active compound are dissolved in 35 parts by weight of an organic solvent (e.g. alkylaromatics) with addition of calcium dodecylbenzenesulfonate and castor oil ethoxylate (in each case 5 parts by weight). This mixture is introduced into 30 parts by weight of water by means of an emulsifier (e.g. Ultraturrax) and made into a homogeneous emulsion. Dilution with water gives an emulsion. The formulation has an active compound content of 25% by weight.

E Suspensions

In an agitated ball mill, 20 parts by weight of active compound are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules

50 parts by weight of active compound are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

G Water-Dispersible Powders and Water-Soluble Powders

75 parts by weight of active compound are ground in a rotor-stator mill with addition of 25 parts by weight of dispersants, welters and silica gel. Dilution with water gives a stable dispersion or solution of the active. compound. The active compound content of the formulation is 75% by weight.

H Gel Formulations

In a ball mill, 20 parts by weight of active compound, 10 parts by weight of dispersant, 1 part by weight of gelling agent and 70 parts by weight of water or of an organic solvent are ground to give a fine suspension. Dilution with water gives a stable suspension with active compound content of 20% by weight.

2. Products to be Applied Undiluted

I Dusts

5 parts by weight of active compound are ground finely and mixed intimately with 95 parts by weight of finely divided kaolin. This gives a dusting powder with an active compound content of 5% by weight.

J Granules (GR, FG, GG, MG)

0.5 parts by weight of active compound are ground finely and associated with 99.5 parts by weight of carriers. Current methods here are extrusion, spray-drying or the fluidized bed. This gives granules to be applied undiluted with an active compound content of 0.5% by weight.

K ULV Solutions (UL)

10 parts by weight of active compound are dissolved in 90 parts by weight of an organic solvent, for example xylene. This gives a product to be applied undiluted with an active compound content of 10% by weight.

The compounds I or the herbicidal compositions comprising them can be applied pre- or post-emergence, or together with the seed of a crop plant. It is also possible to apply the herbicidal compositions or active compounds by applying seed, pretreated with the herbicidal compositions or active compounds, of a crop plant. If the active compounds are less well tolerated by certain crop plants, application techniques may be used in which the herbicidal compositions are sprayed, with the aid of the spraying equipment, in such a way that as far as possible they do not come into contact with the leaves of the sensitive crop plants, while the active compounds reach the leaves of undesirable plants growing underneath, or the bare soil surface (post-directed, lay-by).

In a further embodiment, the compounds of the formula I or the herbicidal compositions can be applied by treating seed.

The treatment of seed comprises essentially all procedures familiar to the person skilled in the art (seed dressing, seed coating, seed dusting, seed soaking, seed film coating, seed multilayer coating, seed encrusting, seed dripping and seed pelleting) based on the compounds of the formula I according to the invention or the compositions prepared therefrom. Here, the herbicidal compositions can be applied diluted or undiluted.

The term seed comprises seed of all types, such as, for example, corns, seeds, fruits, tubers, cuttings and similar forms. Here, preferably, the term seed describes corns and seeds.

The seed used can be seed of the useful plants mentioned above, but also the seed of transgenic plants or plants obtained by customary breeding methods.

The rates of application of active compound are from 0.001 to 3.0, preferably 0.01 to 1.0, kg/ha of active substance (a.s.), depending on the control target, the season, the target plants and the growth stage. To treat the seed, the compounds I are generally employed in amounts of from 0.001 to 10 kg per 100 kg of seed.

It may also be advantageous to use the compounds of the formula I in combination with safeners. Safeners are chemical compounds which prevent or reduce damage to useful plants without substantially affecting the herbicidal action of the compounds of the formula Ion unwanted plants. They can be used both before sowing (for example in the treatment of seed, or on cuttings or seedlings) and before or after the emergence of the useful plant. The safeners and the compounds of the formula I can be used simultaneously or in succession. Suitable safeners are, for example, (quinolin-8-oxy)acetic acids, 1-phenyl-5-haloalkyl-1H-1,2,4-triazole-3-carboxylic acids, 1-phenyl-4,5-dihydro-5-alkyl-1H-pyrazole-3,5-dicarboxylic acids, 4,5-dihydro-5,5-diaryl-3-isoxazolecarboxylic acids, dichloroacetamides, alpha-oximinophenylacetonitriles, acetophenone oximes, 4,6-dihalo-2-phenylpyrimidines, N-[[4-(aminocarbonyl)phenyl]-sulfonyl]-2-benzamides, 1,8-naphthalic anhydride, 2-halo-4-(haloalkyl)-5-thiazole-carboxylic acids, phosphorothiolates and O-phenyl N-alkylcarbamates and their agriculturally useful salts and, provided that they have an acid function, their agriculturally useful derivatives, such as amides, esters and thioesters. To broaden the activity spectrum and to obtain synergistic effects, the compounds of the formula I can be mixed and jointly applied with numerous representatives of other herbicidal or growth-regulating groups of active compounds or with safeners. Suitable mixing partners are, for example, 1,2,4-thiadiazoles, 1,3,4-thiadiazoles, amides, aminophosphoric acid and its derivatives, aminotriazoles, anilides, aryloxy/heteroaryl-oxyalkanoic acids and their derivatives, benzoic acid and its derivatives, benzothiadiazinones, 2-(hetaroyl/aroyl)-1,3-cyclohexanediones, heteroaryl aryl ketones, benzylisoxazolidinones, meta-CF₃-phenyl derivatives, carbamates, quinoline carboxylic acid and its derivatives, chloroacetanilides, cyclohexenone oxime ether derivates, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and heteroaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils: and also phenylpyrazolines and isoxazolines and their derivatives.

Moreover, it may be useful to apply the compounds I alone or in combination with other herbicides or else also mixed with further crop protection agents, jointly, for example with compositions for controlling pests or phytopathogenic fungi or bacteria. Also of interest is the miscibility with mineral salt solutions which are employed for alleviating nutritional and trace element deficiencies. Other additives such as nonphytotoxic oils and oil concentrates may also be added.

Examples of herbicides which can be used in combination with the pyridine compounds of the formula I according to the present invention are:

b1) from the group of the lipid biosynthesis inhibitors:

alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;

b2) from the group of the ALS inhibitors:

amidosulfuron, azimsulfuron, bensulfuron, bensulfuron-methyl, bispyribac, bispyribac-sodium, chlorimuron, chlorimuron-ethyl, chlorsulfuron, cinosulfuron, cloransulam, cloransulam-methyl, cyclosulfamuron, diclosulam, ethametsulfuron, ethametsulfuron-methyl, ethoxysulfuron, flazasulfuron, florasulam, flucarbazone, flucarbazone-sodium, flucetosulfuron, flumetsulam, flupyrsulfuron, flupyrsulfuron-methyl-sodium, foramsulfuron, halosulfuron, halosulfuron-methyl, imazamethabenz, imazamethabenz-methyl, imazamox, imazapic, imazapyr, imazaquin, imazethapyr, imazosulfuron, iodosulfuron, iodosulfuron-methyl-sodium, mesosulfuron, metosulam, metsulfuron, metsulfuron-methyl, nicosulfuron, orthosulfamuron, oxasulfuron, penoxsulam, primisulfuron, primisulfuron-methyl, propoxycarbazone, propoxycarbazone-sodium, prosulfuron, pyrazosulfuron, pyrazosulfuron-ethyl, pyribenzoxim, pyrimisulfan, pyriftalid, pyriminobac, pyriminobac-methyl, pyrithiobac, pyrithiobac-sodium, pyroxsulam, rimsulfuron, sulfometuron, sulfometuron-methyl, sulfosulfuron, thiencarbazone, thiencarbazone-methyl, thifensulfuron, thifensulfuron-methyl, triasulfuron, tribenuron, tribenuron-methyl, trifloxysulfuron, triflusulfuron, triflusulfuron-methyl and tritosulfuron;

b3) from the group of the photosynthesis inhibitors:

ametryn, amicarbazone, atrazine, bentazone, bentazone-sodium, bromacil, bromofenoxim, bromoxynil and its salts and esters, chlorobromuron, chloridazone, chiorotoluron, chioroxuron, cyanazine, desmedipham, desmetryn, dimefuron, dimethametryn, diquat, diquat-dibromide, diuron, fluometuron, hexazinone, ioxynil and its salts and esters, isoproturon, isouron, karbutilate, lenacil, linuron, metamitron, methabenzthiazuron, metobenzuron, metoxuron, metribuzin, monolinuron, neburon, paraquat, paraquat-dichloride, paraquat-dimetilsulfate, pentanochlor, phenmedipham, phenmedipham-ethyl, prometon, prometryn, propanil, propazine, pyridafol, pyridate, siduron, simazine, simetryn, tebuthiuron, terbacil, terbumeton, terbuthylazine, terbutryn, thidiazuron and trietazine;

b4) from the group of the protoporphyrinogen-IX oxidase inhibitors:

acifluorfen, acifluorfen-sodium, azafenidin, bencarbazone, benzfendizone, bifenox, butafenacil, carfentrazone, carfentrazone-ethyl, chiomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, 2-chloro-5-[3,6-dihydro-3-methyl-2,6-dioxo-4-(trifluoromethyl)-1(2H)-pyrimidinyl]-4-fluoro-N-Risopropyl)-methylsulfamoyl]benzamide (B-1; CAS 372137-35-4), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (B-2; CAS 353292-31-6), N-ethyl-3-(2,6-dichloro-4-trifluoro-methylphenoxy)-5-methyl-1H-pyrazole-1-carboxamide (B-3; CAS 452098-92-9), N-tetrahydrofurfuryl-3-(2,6-dichloro-4-trifluoromethylphenoxy)-5-methyl-1H-pyrazole-1-carboxamide (B-4; CAS 915396-43-9), N-ethyl-3-(2-chloro-6-fluoro-4-trifluoromethyl-phenoxy)-5-methyl-1H-pyrazole-1-carboxamide (B-5; CAS 452099-05-7) and N-tetrahydrofurfuryl-3-(2-chloro-6-fluoro-4-trifluoromethylphenoxy)-5-methyl-1H-pyrazole-1-carboxamide (B-6; CAS 45100-03-7);

b5) from the group of the bleacher herbicides:

aclonifen, amitrol, beflubutamid, benzobicyclon, benzofenap, clomazone, diflufenican, fluridone, flurochloridone, flurtamone, isoxaflutole, mesotrione, norflurazon, picolinafen, pyrasulfutole, pyrazolynate, pyrazoxyfen, sulcotrione, tefuryltrione, tembotrione, topramezone, 4-hydroxy-3-[[2-[(2-methoxyethoxy)methyl]-6-(trifluoromethyl)-3-pyridyl]carbonylpicyclo[3.2.1]oct-3-en-2-one (B-7; CAS 352010-68-5) and 4-(3-trifluoromethylphenoxy)-2-(4-trifluoromethylphenyl)pyrimidine (B-8; CAS 180608-33-7);

b6) from the group of the EPSP synthase inhibitors:

glyphosate, glyphosate-isopropylammonium and glyphosate-trimesium (sulfosate);

b7) from the group of the glutamine synthase inhibitors:

bilanaphos (bialaphos), bilanaphos-sodium, glufosinate and glufosinate-ammonium;

b8) from the group of the DHP synthase inhibitors:

asulam;

b9) from the group of the mitose inhibitors:

amiprophos, amiprophos-methyl, benfluralin, butamiphos, butralin, carbetamide, chiorpropham, chlorthal, chlorthal-dimethyl, dinitramine, dithiopyr, ethalfluralin, ftuchloralin, oryzatin, pendtmethatin, prodiamine, propham, propyzamide, tebutam, thiazopyr and trifluralin;

b10) from the group of the VLCFA inhibitors:

acetochior, alachlor, anilofos, butachlor, cafenstrole, dimethachlor, dimethanamid, dimethenamid-P, diphenamid, fentrazamide, flufenacet, mefenacet, metazachlor, metolachlor, metolachlor-S, naproanilide, napropamide, pethoxamid, piperophos, pretilachior, propachlor, propisochlor, pyroxasulfone (KIH-485) and thenylchlor;

Compounds of the formula 2:

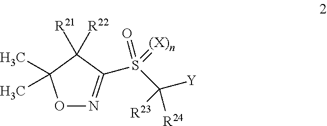

in which the variables have the following meanings:
Y is phenyl or 5- or 6-membered heteroaryl as defined at the outset, which radicals may be substituted by one to three groups R^{aa}; R²¹, R²², R²³, R²⁴ are H, halogen or C₁-C₄-alkyl; X is O or NH; n is 0 or 1.

Compounds of the formula 2 have in particular the following meanings:
Y is

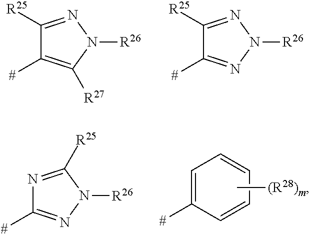

where # denotes the bond to the skeleton of the molecule; and
R²¹, R²², R²³, R²⁴ are H, Cl, F or CH₃; R²⁵ is halogen, C₁-C₄-alkyl or C₁-C₄-haloalkyl; R²⁶ is C₁-C₄-alkyl; R²⁷ is halogen, C₁-C₄-alkoxy or C₁-C₄-haloalkoxy; R²⁸ is H, halogen, C₁-C₄-haloalkyl or C₁-C₄-haloalkoxy; m is 0, 1, 2 or 3; X is oxygen; n is 0 or 1.

Preferred compounds of the formula 2 have the following meanings:
Y

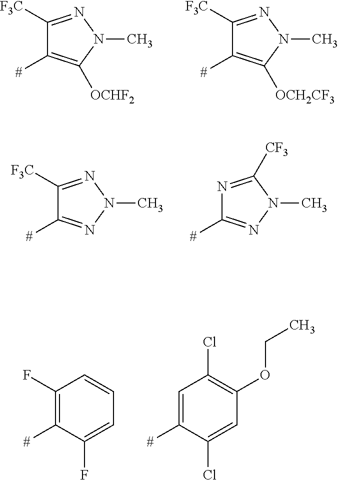

R²¹ is H; R²², R²³ are F; R²⁴ is H or F; X is oxygen; n is 0 or 1.

Particularly preferred compounds of the formula 2 are:
3-[5-(2,2-difluoroethoxy)-1-methyl-3-trifluoromethyl-1H-pyrazol-4-ylmethane-sulfonyl]-4-fluoro-5,5-dimethyl-4,5-dihydroisoxazole (2-1); 3-{[5-(2,2-difluoro-ethoxy)-1-methyl-3-trifluoromethyl-1H-pyrazol-4-yl]fluoromethanesulfonyl}-5,5-dimethyl-4,5-dihydroisoxazole (2-2); 4-(4-fluoro-5,5-dimethyl-4,5-dihydroisoxazole-3-sulfonyl-methyl)-2-methyl-5-trifluoromethyl-2H[1,2,3]triazole (2-3); 4-[(5,5-dimethyl-4,5-dihydroisoxazole-3-sulfonyl)fluoromethyl]-2-methyl-5-trifluoromethyl-2H-[1,2,3]triazole (2-4); 4-(5,5-dimethyl-4,5-dihydroisoxazole-3-sulfonylmethyl)-2-methyl-5-trifluoro-methyl-2H-[1,2,3]triazole (2-5); 3-{[5-(2,2-difluoroethoxy)-1-methyl-3-trifluoromethyl-1H-pyrazol-4-yl]difluoromethanesulfonyl}-5,5-dimethyl-4,5-dihydroisoxazole (2-6); 4-[(5,5-dimethyl-4,5-dihydroisoxazole-3-sulfonyl)difluoromethyl]-2-methyl-5-trifluoro-methyl-2H-[1,2,3]triazole (2-7); 3-{[5-2,2-difluoroethoxy)-1-methyl-3-trifluoromethyl-1H-pyrazol-4-yl]difluoromethanesulfonyl}-4-fluoro-5,5-dimethyl-4,5-dihydroisoxazole (2-8); 4-[difluoro-(4-fluoro-5,5-dimethyl-4,5-dihydroisoxazole-3-sulfonyl)methyl]-2-methyl-5-trifluoromethyl-2H-[1,2,3]triazole (2-9); 3-[(5-difluoromethoxy-1-methyl-3-trifluoromethyl-1H-pyrazol-4-yl)difluoromethanesulfonyl]-5,5-dimethyl-4,5-dihydroisoxazole (2-10);

b11) from the group of the cellulose biosynthesis inhibitors:

chlorthiamid, dichiobenil, flupoxam and isoxaben;

b12) from the group of the decoupler herbicides:

dinoseb, dinoterb and DNOC and its salts;

b13) from the group of the auxin herbicides:

2,4-D and its salts and esters, 2,4-DB and its salts and esters, aminopyralid and its salts such as aminopyralid-tris(2-hydroxypropyl)ammonium and its esters, benazolin, benazolin-ethyl, chioramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, and 5,6-dichloro-2-cyclopropyl-4-pyrimidinecarboxylic acid (B-9; CAS 858956-08-8) and its salts and esters;

b14) from the group of the auxin transport inhibitors: diflufenzopyr, diflufenzopyr-sodium, naptalam and naptalam-sodium;

b15) from the group of the other herbicides: bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, maleic hydrazide, mefluidide, metam, methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3(2-cyclopropyl-6-methylphenoxy)-4-pyridazinol (B-10; CAS 499223-49-3) and its salts and esters.

Examples of preferred safeners C are benoxacor, cloquintocet, cyometrinil, cyprosulfamide, dichiormid, dicyclonone, dietholate, fenchiorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (B-11; MON4660, CAS 71526-07-3) and 2,2,5-trimethyl-3-(dichloroacetyl)-1,3-oxazolidine (B-12; R-29148,
CAS 52836-31-4).

The active compounds of groups b1) to b15) and the safeners C are known herbicides and safeners, see, for example, The Compendium of Pesticide Common Names (http://www.alanwood.net/pesticides/); B. Hock, C. Fedtke, R. R. Schmidt, Herbizide [Herbicides], Georg Thieme Verlag, Stuttgart, 1995. Further herbicidally active compounds are known from WO 96/26202, WO 97/41116, WO 97/41117, WO 97/41118, WO 01/83459 and WO 2008/074991 and from W. Kramer et al. (ed.) “Modern Crop Protection Compounds”, Vol. 1, Wiley VCH, 2007 and the literature quoted therein.

The invention also relates to compositions in the form of a crop protection composition formulated as a 1-component composition comprising an active compound combination comprising at least one pyridine compound of the formula I and at least one further active compound, preferably selected from the active compounds of groups b1 to b15, and at least one solid or liquid carrier and/or one or more surfactants and, if desired, one or more further auxiliaries customary for crop protection compositions.

The invention also relates to compositions in the form of a crop protection composition formulated as a 2-component composition comprising a first component comprising at least one pyridine compound of the formula I, a solid or liquid carrier and/or one or more surfactants and a second component comprising at least one further active compound selected from the active compounds of groups b1 to b15, a solid or liquid carrier and/or one or more surfactants, where additionally both components may also comprise further auxiliaries customary for crop protection compositions.

In binary compositions comprising at least one compound of the formula I as component A and at least one herbicide B, the weight ratio of the active compounds A:B is generally in the range of from 1:1000 to 1000:1, preferably in the range of from 1:500 to 500:1, in particular in the range of from 1:250 to 250:1 and particularly preferably in the range of from 1:75 to 75:1.

In binary compositions comprising at least one compound of the formula I as component A and at least one safener C, the weight ratio of the active compounds A:C is generally in the range of from 1:1000 to 1000:1, preferably in the range of from 1:500 to 500:1, in particular in the range of from 1:250 to 250:1 and particularly preferably in the range of from 1:75 to 75:1.

In ternary compositions comprising both at least one compound of the formula I as component A, at least one herbicide B and at least one safener C, the relative parts by weight of the components A:B are generally in the range of from 1:1000 to 1000:1, preferably in the range of from 1:500 to 500:1, in particular in the range of from 1:250 to 250:1 and particularly preferably in the range of from 1:75 to 75:1; the weight ratio of the components A:C is generally in the range of from 1:1000 to 1000:1, preferably in the range of from 1:500 to 500:1, in particular in the range of from 1:250 to 250:1 and particularly preferably in the range of from 1:75 to 75:1; and the weight ratio of the components B:C is generally in the range of from 1:1000 to 1000:1, preferably in the range of from 1:500 to 500:1, in particular in the range of from 1:250 to 250:1 and particularly preferably in the range of from 1:75 to 75:1. Preferably, the weight ratio of the components A+B to the component C is in the range of from 1:500 to 500:1, in particular in the range of from 1:250 to 250:1 and particularly preferably in the range of from 1:75 to 75:1.

Examples of particularly preferred compositions according to the invention comprising in each case one individualized compound of the formula I and one mixing partner or a mixing partner combination are given in Table B below.

A further aspect of the invention relates to the compositions B-1 to B-1227 listed in Table B below, where in each case one row of Table B corresponds to a herbicidal composition comprising one of the compounds of the formula I individualized in the above description (component 1) and the further active compound from groups b1) to b15) and/or safener C stated in each case in the row in question (component 2). The active compounds in the compositions described are in each case preferably present in synergistically effective amounts.

TABLE B

| | Herbicide(s) B | Safener C |
|---|---|---|
| B-1 | clodinafop-propargyl | — |
| B-2 | cycloxydim | — |
| B-3 | cyhalofop-butyl | — |
| B-4 | fenoxaprop-P-ethyl | — |
| B-5 | pinoxaden | — |
| B-6 | profoxydim | — |
| B-7 | tepraloxydim | — |
| B-8 | tralkoxydim | — |
| B-9 | esprocarb | — |
| B-10 | prosulfocarb | — |
| B-11 | thiobencarb | — |
| B-12 | triallate | — |
| B-13 | bensulfuron-methyl | — |
| B-14 | bispyribac-sodium | — |
| B-15 | cyclosulfamuron | — |
| B-16 | flumetsulam | — |
| B-17 | flupyrsulfuron-methyl-sodium | — |
| B-18 | foramsulfuron | — |
| B-19 | imazamox | — |
| B-20 | imazapic | — |
| B-21 | imazapyr | — |
| B-22 | imazaquin | — |
| B-23 | imazethapyr | — |
| B-24 | imazosulfuron | — |
| B-25 | iodosulfuron-methyl-sodium | — |
| B-26 | mesosulfuron | — |
| B-27 | nicosulfuron | — |
| B-28 | penoxsulam | — |
| B-29 | propoxycarbazone-sodium | — |
| B-30 | pyrazosulfuron-ethyl | — |
| B-31 | pyroxsulam | — |
| B-32 | rimsulfuron | — |
| B-33 | sulfosulfuron | — |
| B-34 | thiencarbazone-methyl | — |
| B-35 | tritosulfuron | — |
| B-36 | 2,4-D and its salts and esters | — |
| B-37 | aminopyralid and its salts and esters | — |
| B-38 | clopyralid and its salts and esters | — |
| B-39 | dicamba and its salts and esters | — |
| B-40 | fluroxypyr-meptyl | — |
| B-41 | quinclorac | — |
| B-42 | quinmerac | — |
| B-43 | B-9 | — |
| B-44 | diflufenzopyr | — |
| B-45 | diflufenzopyr-sodium | — |
| B-46 | clomazone | — |
| B-47 | diflufenican | — |
| B-48 | fluorochloridone | — |
| B-49 | isoxaflutol | — |
| B-50 | mesotrione | — |
| B-51 | picolinafen | — |
| B-52 | sulcotrione | — |
| B-53 | tefuryltrione | — |
| B-54 | tembotrione | — |
| B-55 | topramezone | — |
| B-56 | B-7 | — |
| B-57 | atrazine | — |
| B-58 | diuron | — |
| B-59 | fluometuron | — |
| B-60 | hexazinone | — |
| B-61 | isoproturon | — |
| B-62 | metribuzin | — |
| B-63 | propanil | — |
| B-64 | terbuthylazine | — |
| B-65 | paraquat dichloride | — |
| B-66 | flumioxazin | — |
| B-67 | oxyfluorfen | — |
| B-68 | sulfentrazone | — |
| B-69 | B-1 | — |
| B-70 | B-2 | — |
| B-71 | glyphosate | — |
| B-72 | glyphosate-isopropylammonium | — |
| B-73 | glyphosate-trimesium (sulfosate) | — |
| B-74 | glufosinate | — |
| B-75 | glufosinate-ammonium | — |
| B-76 | pendimethalin | — |
| B-77 | trifluralin | — |
| B-78 | acetochlor | — |
| B-79 | cafenstrole | — |
| B-80 | dimethenamid-P | — |
| B-81 | fentrazamide | — |
| B-82 | flufenacet | — |
| B-83 | mefenacet | — |
| B-84 | metazachlor | — |
| B-85 | metolachlor-S | — |
| B-86 | pyroxasulfone | — |
| B-87 | isoxaben | — |
| B-88 | dymron | — |
| B-89 | indanofan | — |
| B-90 | oxaziclomefone | — |
| B-91 | triaziflam | — |
| B-92 | atrazine + B-1 | — |
| B-93 | atrazine + glyphosate | — |
| B-94 | atrazine + mesotrione | — |
| B-95 | atrazine + nicosulfuron | — |
| B-96 | atrazine + tembotrione | — |
| B-97 | atrazine + topramezone | — |
| B-98 | clomazone + glyphosate | — |
| B-99 | diflufenican + clodinafop-propargyl | — |
| B-100 | diflufenican + fenoxaprop-p-ethyl | — |
| B-101 | diflufenican + flupyrsulfuron-methyl-sodium | — |
| B-102 | diflufenican + glyphosate | — |
| B-103 | diflufenican + mesosulfuron-methyl | — |
| B-104 | diflufenican + pinoxaden | — |
| B-105 | diflufenican + pyroxsulam | — |
| B-106 | flumetsulam + glyphosate | — |
| B-107 | flumioxazin + glyphosate | — |
| B-108 | imazapic + glyphosate | — |
| B-109 | imazethapyr + glyphosate | — |
| B-110 | Isoxaflutol + B-1 | — |
| B-111 | isoxaflutol + glyphosate | — |
| B-112 | metazachlor + B-1 | — |
| B-113 | metazachlor + glyphosate | — |
| B-114 | metazachlor + mesotrione | — |
| B-115 | metazachlor + nicosulfuron | — |
| B-116 | metazachlor + terbuthylazin | — |
| B-117 | metazachlor + topramezone | — |
| B-118 | metribuzin + glyphosate | — |
| B-119 | pendimethalin + B-1 | — |
| B-120 | pendimethalin + clodinafop-propargyl | — |
| B-121 | pendimethalin + fenoxaprop-P-ethyl | — |
| B-122 | pendimethalin + flupyrsulfuron-methyl-sodium | — |
| B-123 | pendimethalin + glyphosate | — |
| B-124 | pendimethalin + mesosulfuron-methyl | — |
| B-125 | pendimethalin + mesotrionee | — |
| B-126 | pendimethalin + nicosulfuron | — |
| B-127 | pendimethalin + pinoxaden | — |
| B-128 | pendimethalin + pyroxsulam | — |
| B-129 | pendimethalin + tembotrione | — |
| B-130 | pendimethalin + topramezone | — |
| B-131 | pyroxasulfone + tembotrione | — |
| B-132 | pyroxasulfone + topramezone | — |
| B-133 | sulfentrazone + glyphosate | — |
| B-134 | terbuthylazin + B-1 | — |
| B-135 | terbuthylazin + foramsulfuron | — |
| B-136 | terbuthylazin + glyphosate | — |
| B-137 | terbuthylazin + mesotrione | — |
| B-138 | terbuthylazin + nicosulfuron | — |
| B-139 | terbuthylazin + tembotrione | — |
| B-140 | terbuthylazin + topramezone | — |
| B-141 | trifluralin + glyphosate | — |
| B-142 | — | benoxacor |
| B-143 | — | cloquintocet |
| B-144 | — | cyprosulfamide |
| B-145 | — | dichlormid |
| B-146 | — | fenchlorazole |
| B-147 | — | isoxadifen |
| B-148 | — | mefenpyr |
| B-149 | — | B-11 |
| B-150 | — | B-12 |
| B-151 | clodinafop-propargyl | benoxacor |
| B-152 | cycloxydim | benoxacor |
| B-153 | cyhalofop-butyl | benoxacor |
| B-154 | fenoxaprop-P-ethyl | benoxacor |
| B-155 | pinoxaden | benoxacor |
| B-156 | profoxydim | benoxacor |
| B-157 | tepraloxydim | benoxacor |
| B-158 | tralkoxydim | benoxacor |
| B-159 | esprocarb | benoxacor |
| B-160 | prosulfocarb | benoxacor |
| B-161 | thiobencarb | benoxacor |
| B-162 | triallate | benoxacor |
| B-163 | bensulfuron-methyl | benoxacor |
| B-164 | bispyribac-sodium | benoxacor |
| B-165 | cyclosulfamuron | benoxacor |
| B-166 | flumetsulam | benoxacor |
| B-167 | flupyrsulfuron-methyl-sodium | benoxacor |
| B-168 | foramsulfuron | benoxacor |
| B-169 | imazamox | benoxacor |
| B-170 | imazapic | benoxacor |
| B-171 | imazapyr | benoxacor |
| B-172 | imazaquin | benoxacor |
| B-173 | imazethapyr | benoxacor |
| B-174 | imazosulfuron | benoxacor |
| B-175 | iodosulfuron-methyl-sodium | benoxacor |
| B-176 | mesosulfuron | benoxacor |
| B-177 | nicosulfuron | benoxacor |
| B-178 | penoxsulam | benoxacor |
| B-179 | propoxycarbazone-sodium | benoxacor |
| B-180 | pyrazosulfuron-ethyl | benoxacor |
| B-181 | pyroxsulam | benoxacor |
| B-182 | rimsulfuron | benoxacor |
| B-183 | sulfosulfuron | benoxacor |
| B-184 | thiencarbazone-methyl | benoxacor |
| B-185 | tritosulfuron | benoxacor |
| B-186 | 2,4-D and its salts and esters | benoxacor |
| B-187 | aminopyralid and its salts and esters | benoxacor |
| B-188 | clopyralid and its salts and esters | benoxacor |
| B-189 | dicamba and its salts and esters | benoxacor |
| B-190 | fluroxypyr-meptyl | benoxacor |
| B-191 | quinclorac | benoxacor |
| B-192 | quinmerac | benoxacor |
| B-193 | B-9 | benoxacor |
| B-194 | diflufenzopyr | benoxacor |
| B-195 | diflufenzopyr-sodium | benoxacor |
| B-196 | clomazone | benoxacor |
| B-197 | diflufenican | benoxacor |
| B-198 | fluorochloridone | benoxacor |
| B-199 | isoxaflutol | benoxacor |
| B-200 | mesotrione | benoxacor |
| B-201 | picolinafen | benoxacor |
| B-202 | sulcotrione | benoxacor |
| B-203 | tefuryltrione | benoxacor |
| B-204 | tembotrione | benoxacor |
| B-205 | topramezone | benoxacor |
| B-206 | B-7 | benoxacor |
| B-207 | atrazine | benoxacor |
| B-208 | diuron | benoxacor |
| B-209 | fluometuron | benoxacor |
| B-210 | hexazinone | benoxacor |
| B-211 | isoproturon | benoxacor |
| B-212 | metribuzin | benoxacor |
| B-213 | propanil | benoxacor |
| B-214 | terbuthylazin | benoxacor |
| B-215 | paraquat dichloride | benoxacor |
| B-216 | flumioxazin | benoxacor |
| B-217 | oxyfluorfen | benoxacor |
| B-218 | sulfentrazone | benoxacor |
| B-219 | B-1 | benoxacor |
| B-220 | B-2 | benoxacor |
| B-221 | glyphosate | benoxacor |
| B-222 | glyphosate-isopropylammonium | benoxacor |
| B-223 | glyphosate-trimesium (sulfosate) | benoxacor |
| B-224 | glufosinate | benoxacor |
| B-225 | glufosinate-ammonium | benoxacor |
| B-226 | pendimethalin | benoxacor |
| B-227 | trifluralin | benoxacor |
| B-228 | acetochlor | benoxacor |
| B-229 | cafenstrole | benoxacor |
| B-230 | dimethenamid-P | benoxacor |
| B-231 | fentrazamide | benoxacor |
| B-232 | flufenacet | benoxacor |
| B-233 | mefenacet | benoxacor |
| B-234 | metazachlor | benoxacor |
| B-235 | metolachlor-S | benoxacor |
| B-236 | pyroxasulfone | benoxacor |
| B-237 | isoxaben | benoxacor |
| B-238 | dymron | benoxacor |
| B-239 | indanofan | benoxacor |
| B-240 | oxaziclomefone | benoxacor |
| B-241 | triaziflam | benoxacor |
| B-242 | atrazine + B-1 | benoxacor |
| B-243 | atrazine + glyphosate | benoxacor |
| B-244 | atrazine + mesotrione | benoxacor |
| B-245 | atrazine + nicosulfuron | benoxacor |
| B-246 | atrazine + tembotrione | benoxacor |
| B-247 | atrazine + topramezone | benoxacor |
| B-248 | clomazone + glyphosate | benoxacor |
| B-249 | diflufenican + clodinafop-propargyl | benoxacor |
| B-250 | diflufenican + fenoxaprop-P-ethyl | benoxacor |
| B-251 | diflufenican + flupyrsulfuron-methyl-sodium | benoxacor |
| B-252 | diflufenican + glyphosate | benoxacor |
| B-253 | diflufenican + mesosulfuron-methyl | benoxacor |
| B-254 | diflufenican + pinoxaden | benoxacor |
| B-255 | diflufenican + pyroxsulam | benoxacor |
| B-256 | flumetsulam + glyphosate | benoxacor |
| B-257 | flumioxazin + glyphosate | benoxacor |
| B-258 | imazapic + glyphosate | benoxacor |
| B-259 | imazethapyr + glyphosate | benoxacor |
| B-260 | isoxaflutol + B-1 | benoxacor |
| B-261 | isoxaflutol + glyphosate | benoxacor |
| B-262 | metazachlor + B-1 | benoxacor |
| B-263 | metazachlor + glyphosate | benoxacor |
| B-264 | metazachlor + mesotrione | benoxacor |
| B-265 | metazachlor + nicosulfuron | benoxacor |
| B-266 | metazachlor + terbuthylazin | benoxacor |
| B-267 | metazachlor + topramezone | benoxacor |
| B-268 | metribuzin + glyphosate | benoxacor |
| B-269 | pendimethalin + B-1 | benoxacor |
| B-270 | pendimethalin + clodinafop-propargyl | benoxacor |
| B-271 | pendimethalin + fenoxaprop-P-ethyl | benoxacor |
| B-272 | pendimethalin + flupyrsulfuron-methyl-sodium | benoxacor |
| B-273 | pendimethalin + glyphosate | benoxacor |
| B-274 | pendimethalin + mesosulfuron-methyl | benoxacor |
| B-275 | pendimethalin + mesotrionee | benoxacor |
| B-276 | pendimethalin + nicosulfuron | benoxacor |
| B-277 | pendimethalin + pinoxaden | benoxacor |
| B-278 | pendimethalin + pyroxsulam | benoxacor |
| B-279 | pendimethalin + tembotrione | benoxacor |
| B-280 | pendimethalin + topramezone | benoxacor |
| B-281 | pyroxasulfone + tembotrione | benoxacor |
| B-282 | pyroxasulfone + topramezone | benoxacor |
| B-283 | sulfentrazone + glyphosate | benoxacor |
| B-284 | terbuthylazin + B-1 | benoxacor |
| B-285 | terbuthylazin + foramsulfuron | benoxacor |
| B-286 | terbuthylazin + glyphosate | benoxacor |
| B-287 | terbuthylazin + mesotrione | benoxacor |
| B-288 | terbuthylazin + nicosulfuron | benoxacor |
| B-289 | terbuthylazin + tembotrione | benoxacor |
| B-290 | terbuthylazin + topramezone | benoxacor |
| B-291 | trifluralin + glyphosate | benoxacor |
| B-292 | clodinafop-propargyl | cloquintocet |
| B-293 | cycloxydim | cloquintocet |
| B-294 | cyhalofop-butyl | cloquintocet |
| B-295 | fenoxaprop-P-ethyl | cloquintocet |
| B-296 | pinoxaden | cloquintocet |
| B-297 | profoxydim | cloquintocet |
| B-298 | tepraloxydim | cloquintocet |
| B-299 | tralkoxydim | cloquintocet |
| B-300 | esprocarb | cloquintocet |
| B-301 | prosulfocarb | cloquintocet |
| B-302 | thiobencarb | cloquintocet |
| B-303 | triallate | cloquintocet |
| B-304 | bensulfuron-methyl | cloquintocet |
| B-305 | bispyribac-sodium | cloquintocet |
| B-306 | cyclosulfamuron | cloquintocet |
| B-307 | flumetsulam | cloquintocet |
| B-308 | flupyrsulfuron-methyl-sodium | cloquintocet |
| B-309 | foramsulfuron | cloquintocet |
| B-310 | imazamox | cloquintocet |
| B-311 | imazapic | cloquintocet |
| B-312 | imazapyr | cloquintocet |
| B-313 | imazaquin | cloquintocet |
| B-314 | imazethapyr | cloquintocet |
| B-315 | imazosulfuron | cloquintocet |
| B-316 | iodosulfuron-methyl-sodium | cloquintocet |
| B-317 | mesosulfuron | cloquintocet |
| B-318 | nicosulfuron | cloquintocet |
| B-319 | penoxsulam | cloquintocet |
| B-320 | propoxycarbazone-sodium | cloquintocet |
| B-321 | pyrazosulfuron-ethyl | cloquintocet |
| B-322 | pyroxsulam | cloquintocet |
| B-323 | rimsulfuron | cloquintocet |
| B-324 | sulfosulfuron | cloquintocet |
| B-325 | thiencarbazone-methyl | cloquintocet |
| B-326 | tritosulfuron | cloquintocet |
| B-327 | 2,4-D and its salts and esters | cloquintocet |
| B-328 | aminopyralid and its salts and esters | cloquintocet |
| B-329 | clopyralid and its salts and esters | cloquintocet |
| B-330 | dicamba and its salts and esters | cloquintocet |
| B-331 | fluroxypyr-meptyl | cloquintocet |
| B-332 | quinclorac | cloquintocet |
| B-333 | quinmerac | cloquintocet |
| B-334 | B-9 | cloquintocet |
| B-335 | diflufenzopyr | cloquintocet |
| B-336 | diflufenzopyr-sodium | cloquintocet |
| B-337 | clomazone | cloquintocet |
| B-338 | diflufenican | cloquintocet |
| B-339 | fluorochloridone | cloquintocet |
| B-340 | isoxaflutol | cloquintocet |
| B-341 | mesotrione | cloquintocet |
| B-342 | picolinafen | cloquintocet |
| B-343 | sulcotrione | cloquintocet |
| B-344 | tefuryltrione | cloquintocet |
| B-345 | tembotrione | cloquintocet |
| B-346 | topramezone | cloquintocet |
| B-347 | B-7 | cloquintocet |
| B-348 | atrazine | cloquintocet |
| B-349 | diuron | cloquintocet |
| B-350 | fluometuron | cloquintocet |
| B-351 | hexazinone | cloquintocet |
| B-352 | isoproturon | cloquintocet |
| B-353 | metribuzin | cloquintocet |
| B-354 | propanil | cloquintocet |
| B-355 | terbuthylazin | cloquintocet |
| B-356 | paraquat dichloride | cloquintocet |
| B-357 | flumioxazin | cloquintocet |
| B-358 | oxyfluorfen | cloquintocet |
| B-359 | sulfentrazone | cloquintocet |
| B-360 | B-1 | cloquintocet |
| B-361 | B-2 | cloquintocet |
| B-362 | glyphosate | cloquintocet |
| B-363 | glyphosate-isopropylammonium | cloquintocet |
| B-364 | glyphosate-trimesium (sulfosate) | cloquintocet |
| B-365 | glufosinate | cloquintocet |
| B-366 | glufosinate-ammonium | cloquintocet |
| B-367 | pendimethalin | cloquintocet |
| B-368 | trifluralin | cloquintocet |
| B-369 | acetochlor | cloquintocet |
| B-370 | cafenstrole | cloquintocet |
| B-371 | dimethenamid-P | cloquintocet |
| B-372 | fentrazamide | cloquintocet |
| B-373 | flufenacet | cloquintocet |
| B-374 | mefenacet | cloquintocet |
| B-375 | metazachlor | cloquintocet |
| B-376 | metolachlor-S | cloquintocet |
| B-377 | pyroxasulfone | cloquintocet |
| B-378 | isoxaben | cloquintocet |
| B-379 | dymron | cloquintocet |
| B-380 | indanofan | cloquintocet |
| B-381 | oxaziclomefone | cloquintocet |
| B-382 | triaziflam | cloquintocet |
| B-383 | atrazine + B-1 | cloquintocet |
| B-384 | atrazine + glyphosate | cloquintocet |
| B-385 | atrazine + mesotrione | cloquintocet |
| B-386 | atrazine + nicosulfuron | cloquintocet |
| B-387 | atrazine + tembotrione | cloquintocet |
| B-388 | atrazine + topramezone | cloquintocet |
| B-389 | clomazone + glyphosate | cloquintocet |
| B-390 | diflufenican + clodinafop-propargyl | cloquintocet |
| B-391 | diflufenican + fenoxaprop-p-ethyl | cloquintocet |
| B-392 | diflufenican + flupyrsulfuron-methyl-sodium | cloquintocet |
| B-393 | diflufenican + glyphosate | cloquintocet |
| B-394 | diflufenican + mesosulfuron-methyl | cloquintocet |
| B-395 | diflufenican + pinoxaden | cloquintocet |
| B-396 | diflufenican + pyroxsulam | cloquintocet |
| B-397 | flumetsulam + glyphosate | cloquintocet |
| B-398 | flumioxazin + glyphosate | cloquintocet |
| B-399 | imazapic + glyphosate | cloquintocet |
| B-400 | imazethapyr + glyphosate | cloquintocet |
| B-401 | isoxaflutol + B-1 | cloquintocet |
| B-402 | isoxaflutol + glyphosate | cloquintocet |
| B-403 | metazachlor + B-1 | cloquintocet |
| B-404 | metazachlor + glyphosate | cloquintocet |
| B-405 | metazachlor + mesotrione | cloquintocet |
| B-406 | metazachlor + nicosulfuron | cloquintocet |
| B-407 | metazachlor + terbuthylazin | cloquintocet |
| B-408 | metazachlor + topramezone | cloquintocet |
| B-409 | metribuzin + glyphosate | cloquintocet |
| B-410 | pendimethalin + B-1 | cloquintocet |
| B-411 | pendimethalin + clodinafop-propargyl | cloquintocet |
| B-412 | pendimethalin + fenoxaprop-P-ethyl | cloquintocet |
| B-413 | pendimethalin + flupyrsulfuron-methyl-sodium | cloquintocet |
| B-414 | pendimethalin + glyphosate | cloquintocet |
| B-415 | pendimethalin + mesosulfuron-methyl | cloquintocet |
| B-416 | pendimethalin + mesotrione | cloquintocet |
| B-417 | pendimethalin + nicosulfuron | cloquintocet |
| B-418 | pendimethalin + pinoxaden | cloquintocet |
| B-419 | pendimethalin + pyroxsulam | cloquintocet |
| B-420 | pendimethalin + tembotrione | cloquintocet |
| B-421 | pendimethalin + topramezone | cloquintocet |
| B-422 | pyroxasulfone + tembotrione | cloquintocet |
| B-423 | pyroxasulfone + topramezone | cloquintocet |
| B-424 | sulfentrazone + glyphosate | cloquintocet |
| B-425 | terbuthylazin + B-1 | cloquintocet |
| B-426 | terbuthylazin + foramsulfuron | cloquintocet |
| B-427 | terbuthylazin + glyphosate | cloquintocet |
| B-428 | terbuthylazin + mesotrione | cloquintocet |
| B-429 | terbuthylazin + nicosulfuron | cloquintocet |
| B-430 | terbuthylazin + tembotrione | cloquintocet |
| B-431 | terbuthylazin + topramezone | cloquintocet |
| B-432 | trifluralin + glyphosate | cloquintocet |
| B-433 | clodinafop-propargyl | dichlormid |
| B-434 | cycloxydim | dichlormid |
| B-435 | cyhalofop-butyl | dichlormid |
| B-436 | fenoxaprop-P-ethyl | dichlormid |
| B-437 | pinoxaden | dichlormid |
| B-438 | profoxydim | dichlormid |
| B-439 | tepraloxydim | dichlormid |
| B-440 | tralkoxydim | dichlormid |
| B-441 | esprocarb | dichlormid |
| B-442 | prosulfocarb | dichlormid |
| B-443 | thiobencarb | dichlormid |
| B-444 | triallate | dichlormid |
| B-445 | bensulfuron-methyl | dichlormid |
| B-446 | bispyribac-sodium | dichlormid |
| B-447 | cyclosulfamuron | dichlormid |
| B-448 | flumetsulam | dichlormid |
| B-449 | flupyrsulfuron-methyl-sodium | dichlormid |
| B-450 | foramsulfuron | dichlormid |
| B-451 | imazamox | dichlormid |
| B-452 | imazapic | dichlormid |
| B-453 | imazapyr | dichlormid |
| B-454 | imazaquin | dichlormid |
| B-455 | imazethapyr | dichlormid |
| B-456 | imazosulfuron | dichlormid |
| B-457 | iodosulfuron-methyl-sodium | dichlormid |
| B-458 | mesosulfuron | dichlormid |
| B-459 | nicosulfuron | dichlormid |
| B-460 | penoxsulam | dichlormid |
| B-461 | propoxycarbazone-sodium | dichlormid |
| B-462 | pyrazosulfuron-ethyl | dichlormid |
| B-463 | pyroxsulam | dichlormid |
| B-464 | rimsulfuron | dichlormid |
| B-465 | sulfosulfuron | dichlormid |
| B-466 | thiencarbazone-methyl | dichlormid |
| B-467 | tritosulfuron | dichlormid |
| B-468 | 2,4-D and its salts and esters | dichlormid |
| B-469 | aminopyralid and its salts and esters | dichlormid |
| B-470 | clopyralid and its salts and esters | dichlormid |
| B-471 | dicamba and its salts and esters | dichlormid |
| B-472 | fluroxypyr-meptyl | dichlormid |
| B-473 | quinclorac | dichlormid |
| B-474 | quinmerac | dichlormid |
| B-475 | B-9 | dichlormid |
| B-476 | diflufenzopyr | dichlormid |
| B-477 | diflufenzopyr-sodium | dichlormid |
| B-478 | clomazone | dichlormid |
| B-479 | diflufenican | dichlormid |
| B-480 | fluorochloridone | dichlormid |
| B-481 | isoxaflutol | dichlormid |
| B-482 | mesotrione | dichlormid |
| B-483 | picolinafen | dichlormid |
| B-484 | sulcotrione | dichlormid |
| B-485 | tefuryltrione | dichlormid |
| B-486 | tembotrione | dichlormid |
| B-487 | topramezone | dichlormid |
| B-488 | B-7 | dichlormid |
| B-489 | atrazine | dichlormid |
| B-490 | diuron | dichlormid |
| B-491 | fluometuron | dichlormid |
| B-492 | hexazinone | dichlormid |
| B-493 | isoproturon | dichlormid |
| B-494 | metribuzin | dichlormid |
| B-495 | propanil | dichlormid |
| B-496 | terbuthylazin | dichlormid |
| B-497 | paraquat dichloride | dichlormid |
| B-498 | flumioxazin | dichlormid |
| B-499 | oxyfluorfen | dichlormid |
| B-500 | sulfentrazone | dichlormid |
| B-501 | B-1 | dichlormid |
| B-502 | B-2 | dichlormid |
| B-503 | glyphosate | dichlormid |
| B-504 | glyphosate-isopropylammonium | dichlormid |
| B-505 | glyphosate-trimesium (sulfosate) | dichlormid |
| B-506 | glufosinate | dichlormid |
| B-507 | glufosinate-ammonium | dichlormid |
| B-508 | pendimethalin | dichlormid |
| B-509 | trifluralin | dichlormid |
| B-510 | acetochlor | dichlormid |
| B-511 | cafenstrole | dichlormid |
| B-512 | dimethenamid-P | dichlormid |
| B-513 | fentrazamide | dichlormid |
| B-514 | flufenacet | dichlormid |
| B-515 | mefenacet | dichlormid |
| B-516 | metazachlor | dichlormid |
| B-517 | metolachlor-S | dichlormid |
| B-518 | pyroxasulfone | dichlormid |
| B-519 | isoxaben | dichlormid |
| B-520 | dymron | dichlormid |
| B-521 | indanofan | dichlormid |
| B-522 | oxaziclomefone | dichlormid |
| B-523 | triaziflam | dichlormid |
| B-524 | atrazine + B-1 | dichlormid |
| B-525 | atrazine + glyphosate | dichlormid |
| B-526 | atrazine + mesotrione | dichlormid |
| B-527 | atrazine + nicosulfuron | dichlormid |
| B-528 | atrazine + tembotrione | dichlormid |
| B-529 | atrazine + topramezone | dichlormid |
| B-530 | clomazone + glyphosate | dichlormid |
| B-531 | diflufenican + clodinafop-propargyl | dichlormid |
| B-532 | diflufenican + fenoxaprop-p-ethyl | dichlormid |
| B-533 | diflufenican + flupyrsulfuron-methyl-sodium | dichlormid |
| B-534 | diflufenican + glyphosate | dichlormid |
| B-535 | diflufenican + mesosulfuron-methyl | dichlormid |
| B-536 | diflufenican + pinoxaden | dichlormid |
| B-537 | diflufenican + pyroxsulam | dichlormid |
| B-538 | flumetsulam + glyphosate | dichlormid |
| B-539 | flumioxazin + glyphosate | dichlormid |
| B-540 | imazapic + glyphosate | dichlormid |
| B-541 | imazethapyr + glyphosate | dichlormid |
| B-542 | isoxaflutol + B-1 | dichlormid |
| B-543 | isoxaflutol + glyphosate | dichlormid |
| B-544 | metazachlor + B-1 | dichlormid |
| B-545 | metazachlor + glyphosate | dichlormid |
| B-546 | metazachlor + mesotrione | dichlormid |
| B-547 | metazachlor + nicosulfuron | dichlormid |
| B-548 | metazachlor + terbuthylazin | dichlormid |
| B-549 | metazachlor + topramezone | dichlormid |
| B-550 | metribuzin + glyphosate | dichlormid |
| B-551 | pendimethalin + B-1 | dichlormid |
| B-552 | pendimethalin + clodinafop-propargyl | dichlormid |
| B-553 | pendimethalin + fenoxaprop-P-ethyl | dichlormid |
| B-554 | pendimethalin + flupyrsulfuron-methyl-sodium | dichlormid |
| B-555 | pendimethalin + glyphosate | dichlormid |
| B-556 | pendimethalin + mesosulfuron-methyl | dichlormid |
| B-557 | pendimethalin + mesotrione | dichlormid |
| B-558 | pendimethalin + nicosulfuron | dichlormid |
| B-559 | pendimethalin + pinoxaden | dichlormid |
| B-560 | pendimethalin + pyroxsulam | dichlormid |
| B-561 | pendimethalin + tembotrione | dichlormid |
| B-562 | pendimethalin + topramezone | dichlormid |
| B-563 | pyroxasulfone + tembotrione | dichlormid |
| B-564 | pyroxasulfone + topramezone | dichlormid |
| B-565 | sulfentrazone + glyphosate | dichlormid |
| B-566 | ferbuthylazin + B-1 | dichlormid |
| B-567 | terbuthylazin + foramsulfuron | dichlormid |
| B-568 | terbuthylazin + glyphosate | dichlormid |
| B-569 | terbuthylazin + mesotrione | dichlormid |
| B-570 | terbuthylazin + nicosulfuron | dichlormid |
| B-571 | terbuthylazin + tembotrione | dichlormid |
| B-572 | terbuthylazin + topramezone | dichlormid |
| B-573 | trifluralin + glyphosate | dichlormid |
| B-574 | clodinafop-propargyl | fenchlorazole |
| B-575 | cycloxydim | fenchlorazole |
| B-576 | cyhalofop-butyl | fenchlorazole |
| B-577 | fenoxaprop-P-ethyl | fenchlorazole |
| B-578 | pinoxaden | fenchlorazole |
| B-579 | profoxydim | fenchlorazole |
| B-580 | tepraloxydim | fenchlorazole |
| B-581 | tralkoxydim | fenchlorazole |
| B-582 | esprocarb | fenchlorazole |
| B-583 | prosulfocarb | fenchlorazole |
| B-584 | thiobencarb | fenchlorazole |
| B-585 | triallate | fenchlorazole |
| B-586 | bensulfuron-methyl | fenchlorazole |
| B-587 | bispyribac-sodium | fenchlorazole |
| B-588 | cyclosulfamuron | fenchlorazole |
| B-589 | flumetsulam | fenchlorazole |
| B-590 | flupyrsulfuron-methyl-sodium | fenchlorazole |
| B-591 | foramsulfuron | fenchlorazole |
| B-592 | imazamox | fenchlorazole |
| B-593 | imazapic | fenchlorazole |
| B-594 | imazapyr | fenchlorazole |
| B-595 | imazaquin | fenchlorazole |
| B-596 | imazethapyr | fenchlorazole |
| B-597 | imazosulfuron | fenchlorazole |
| B-598 | iodosulfuron-methyl-sodium | fenchlorazole |
| B-599 | mesosulfuron | fenchlorazole |
| B-600 | nicosulfuron | fenchlorazole |
| B-601 | penoxsulam | fenchlorazole |
| B-602 | propoxycarbazone-sodium | fenchlorazole |
| B-603 | pyrazosulfuron-ethyl | fenchlorazole |
| B-604 | pyroxsulam | fenchlorazole |
| B-605 | rimsulfuron | fenchlorazole |
| B-606 | sulfosulfuron | fenchlorazole |
| B-607 | thiencarbazone-methyl | fenchlorazole |
| B-608 | tritosulfuron | fenchlorazole |
| B-609 | 2,4-D and its salts and esters | fenchlorazole |
| B-610 | aminopyralid and its salts and esters | fenchlorazole |
| B-611 | clopyralid and its salts and esters | fenchlorazole |
| B-612 | dicamba and its salts and esters | fenchlorazole |
| B-613 | fluroxypyr-meptyl | fenchlorazole |
| B-614 | quinclorac | fenchlorazole |
| B-615 | quinmerac | fenchlorazole |
| B-616 | B-9 | fenchlorazole |
| B-617 | diflufenzopyr | fenchlorazole |
| B-618 | diflufenzopyr-sodium | fenchlorazole |
| B-619 | clomazone | fenchlorazole |
| B-620 | diflufenican | fenchlorazole |
| B-621 | fluorochloridone | fenchlorazole |
| B-622 | isoxaflutol | fenchlorazole |
| B-623 | mesotrione | fenchlorazole |
| B-624 | picolinafen | fenchlorazole |
| B-625 | sulcotrione | fenchlorazole |
| B-626 | tefuryltrione | fenchlorazole |
| B-627 | tembotrione | fenchlorazole |
| B-628 | topramezone | fenchlorazole |
| B-629 | B-7 | fenchlorazole |
| B-630 | atrazine | fenchlorazole |
| B-631 | diuron | fenchlorazole |
| B-632 | fluometuron | fenchlorazole |
| B-633 | hexazinone | fenchlorazole |
| B-634 | isoproturon | fenchlorazole |
| B-635 | metribuzin | fenchlorazole |
| B-636 | propanil | fenchlorazole |
| B-637 | terbuthylazin | fenchlorazole |
| B-638 | paraquat dichloride | fenchlorazole |
| B-639 | flumioxazin | fenchlorazole |
| B-640 | oxyfluorfen | fenchlorazole |
| B-641 | sulfentrazone | fenchlorazole |
| B-642 | B-1 | fenchlorazole |
| B-643 | B-2 | fenchlorazole |
| B-644 | glyphosate | fenchlorazole |
| B-645 | glyphosate-isopropylammonium | fenchlorazole |
| B-646 | glyphosate-trimesium (sulfosate) | fenchlorazole |
| B-647 | glufosinate | fenchlorazole |
| B-648 | glufosinate-ammonium | fenchlorazole |
| B-649 | pendimethalin | fenchlorazole |
| B-650 | trifluralin | fenchlorazole |
| B-651 | acetochlor | fenchlorazole |
| B-652 | cafenstrole | fenchlorazole |
| B-653 | dimethenamid-P | fenchlorazole |
| B-654 | fentrazamide | fenchlorazole |
| B-655 | flufenacet | fenchlorazole |
| B-656 | mefenacet | fenchlorazole |
| B-657 | metazachlor | fenchlorazole |
| B-658 | metolachlor-S | fenchlorazole |
| B-659 | pyroxasulfone | fenchlorazole |
| B-660 | isoxaben | fenchlorazole |
| B-661 | dymron | fenchlorazole |
| B-662 | indanofan | fenchlorazole |
| B-663 | oxaziclomefone | fenchlorazole |
| B-664 | friaziflam | fenchlorazole |
| B-665 | atrazine + B-1 | fenchlorazole |
| B-666 | atrazine + glyphosate | fenchlorazole |
| B-667 | atrazine + mesotrione | fenchlorazole |
| B-668 | atrazine + nicosulfuron | fenchlorazole |
| B-669 | atrazine + tembotrione | fenchlorazole |
| B-670 | atrazine + topramezone | fenchlorazole |
| B-671 | clomazone + glyphosate | fenchlorazole |
| B-672 | diflufenican + clodinafop-propargyl | fenchlorazole |
| B-673 | diflufenican + fenoxaprop-P-ethyl | fenchlorazole |
| B-674 | diflufenican + flupyrsulfuron-methyl-sodium | fenchlorazole |
| B-675 | diflufenican + glyphosate | fenchlorazole |
| B-676 | diflufenican + mesosulfuron-methyl | fenchlorazole |
| B-677 | diflufenican + pinoxaden | fenchlorazole |
| B-678 | diflufenican + pyroxsulam | fenchlorazole |
| B-679 | flumetsulam + glyphosate | fenchlorazole |
| B-680 | flumioxazin + glyphosate | fenchlorazole |
| B-681 | imazapic + glyphosate | fenchlorazole |
| B-682 | imazethapyr + glyphosate | fenchlorazole |
| B-683 | isoxaflutol + B-1 | fenchlorazole |
| B-684 | isoxaflutol + glyphosate | fenchlorazole |
| B-685 | metazachlor + B-1 | fenchlorazole |
| B-686 | metazachlor + glyphosate | fenchlorazole |
| B-687 | metazachlor + mesotrione | fenchlorazole |
| B-688 | metazachlor + nicosulfuron | fenchlorazole |
| B-689 | metazachlor + terbuthylazin | fenchlorazole |
| B-690 | metazachlor + topramezone | fenchlorazole |
| B-691 | metribuzin + glyphosate | fenchlorazole |
| B-692 | pendimethalin + B-1 | fenchlorazole |
| B-693 | pendimethalin + clodinafop-propargyl | fenchlorazole |
| B-694 | pendimethalin + fenoxaprop-P-ethyl | fenchlorazole |
| B-695 | pendimethalin + flupyrsulfuron-methyl-sodium | fenchlorazole |
| B-696 | pendimethalin + glyphosate | fenchlorazole |
| B-697 | pendimethalin + mesosulfuron-methyl | fenchlorazole |
| B-698 | pendimethalin + mesotrione | fenchlorazole |
| B-699 | pendimethalin + nicosulfuron | fenchlorazole |
| B-700 | pendimethalin + pinoxaden | fenchlorazole |
| B-701 | pendimethalin + pyroxsulam | fenchlorazole |
| B-702 | pendimethalin + tembotrione | fenchlorazole |
| B-703 | pendimethalin + topramezone | fenchlorazole |
| B-704 | pyroxasulfone + tembotrione | fenchlorazole |
| B-705 | pyroxasulfone + topramezone | fenchlorazole |
| B-706 | sulfentrazone + glyphosate | fenchlorazole |
| B-707 | terbuthylazin + B-1 | fenchlorazole |
| B-708 | terbuthylazin + foramsulfuron | fenchlorazole |
| B-709 | terbuthylazin + glyphosate | fenchlorazole |
| B-710 | terbuthylazin + mesotrione | fenchlorazole |
| B-711 | terbuthylazin + nicosulfuron | fenchlorazole |
| B-712 | terbuthylazin + tembotrione | fenchlorazole |
| B-713 | terbuthylazin + topramezone | fenchlorazole |
| B-714 | trifluralin + glyphosate | fenchlorazole |
| B-715 | clodinafop-propargyl | isoxadifen |
| B-716 | cycloxydim | isoxadifen |
| B-717 | cyhalofop-butyl | isoxadifen |
| B-718 | fenoxaprop-P-ethyl | isoxadifen |
| B-719 | pinoxaden | isoxadifen |
| B-720 | profoxydim | isoxadifen |
| B-721 | tepraloxydim | isoxadifen |
| B-722 | tralkoxydim | isoxadifen |
| B-723 | esprocarb | isoxadifen |
| B-724 | prosulfocarb | isoxadifen |
| B-725 | thiobencarb | isoxadifen |
| B-726 | triallate | isoxadifen |
| B-727 | bensulfuron-methyl | isoxadifen |
| B-728 | bispyribac-sodium | isoxadifen |
| B-729 | cyclosulfamuron | isoxadifen |
| B-730 | flumetsulam | isoxadifen |
| B-731 | flupyrsulfuron-methyl-sodium | isoxadifen |
| B-732 | foramsulfuron | isoxadifen |
| B-733 | imazamox | isoxadifen |
| B-734 | imazapic | isoxadifen |
| B-735 | imazapyr | isoxadifen |
| B-736 | imazaquin | isoxadifen |
| B-737 | imazethapyr | isoxadifen |
| B-738 | imazosulfuron | isoxadifen |
| B-739 | iodosulfuron-methyl-sodium | isoxadifen |
| B-740 | mesosulfuron | isoxadifen |
| B-741 | nicosulfuron | isoxadifen |
| B-742 | penoxsulam | isoxadifen |
| B-743 | propoxycarbazone-sodium | isoxadifen |
| B-744 | pyrazosulfuron-ethyl | isoxadifen |
| B-745 | pyroxsulam | isoxadifen |
| B-746 | rimsulfuron | isoxadifen |
| B-747 | sulfosulfuron | isoxadifen |
| B-748 | thiencarbazone-methyl | isoxadifen |
| B-749 | tritosulfuron | isoxadifen |
| B-750 | 2,4-D and its salts and esters | isoxadifen |
| B-751 | aminopyralid and its salts and esters | isoxadifen |
| B-752 | clopyralid and its salts and esters | isoxadifen |
| B-753 | dicamba and its salts and esters | isoxadifen |
| B-754 | fluroxypyr-meptyl | isoxadifen |
| B-755 | quinclorac | isoxadifen |
| B-756 | quinmerac | isoxadifen |
| B-757 | B-9 | isoxadifen |
| B-758 | diflufenzopyr | isoxadifen |
| B-759 | diflufenzopyr-sodium | isoxadifen |
| B-760 | clomazone | isoxadifen |
| B-761 | diflufenican | isoxadifen |
| B-762 | fluorochloridone | isoxadifen |
| B-763 | isoxaflutol | isoxadifen |
| B-764 | mesotrione | isoxadifen |
| B-765 | picolinafen | isoxadifen |
| B-766 | sulcotrione | isoxadifen |
| B-767 | tefuryltrione | isoxadifen |
| B-768 | tembotrione | isoxadifen |
| B-769 | topramezone | isoxadifen |
| B-770 | B-7 | isoxadifen |
| B-771 | atrazine | isoxadifen |
| B-772 | diuron | isoxadifen |
| B-773 | fluometuron | isoxadifen |
| B-774 | hexazinone | isoxadifen |
| B-775 | isoproturon | isoxadifen |
| B-776 | metribuzin | isoxadifen |
| B-777 | propanil | isoxadifen |
| B-778 | terbuthylazin | isoxadifen |
| B-779 | paraquat dichloride | isoxadifen |
| B-780 | flumioxazin | isoxadifen |
| B-781 | oxyfluorfen | isoxadifen |
| B-782 | sulfentrazone | isoxadifen |
| B-783 | B-1 | isoxadifen |
| B-784 | B-2 | isoxadifen |
| B-785 | glyphosate | isoxadifen |
| B-786 | glyphosate-isopropylammonium | isoxadifen |
| B-787 | glyphosate-trimesium (sulfosate) | isoxadifen |
| B-788 | glufosinate | isoxadifen |
| B-789 | glufosinate-ammonium | isoxadifen |
| B-790 | pendimethalin | isoxadifen |
| B-791 | trifluralin | isoxadifen |
| B-792 | acetochlor | isoxadifen |
| B-793 | cafenstrole | isoxadifen |
| B-794 | dimethenamid-P | isoxadifen |
| B-795 | fentrazamide | isoxadifen |
| B-796 | flufenacet | isoxadifen |
| B-797 | mefenacet | isoxadifen |
| B-798 | metazachlor | isoxadifen |
| B-799 | metolachlor-S | isoxadifen |
| B-800 | pyroxasulfone | isoxadifen |
| B-801 | isoxaben | isoxadifen |
| B-802 | dymron | isoxadifen |
| B-803 | indanofan | isoxadifen |
| B-804 | oxaziclomefone | isoxadifen |
| B-805 | triaziflam | isoxadifen |
| B-806 | atrazine + B-1 | isoxadifen |
| B-807 | atrazine + glyphosate | isoxadifen |
| B-808 | atrazine + mesotrione | isoxadifen |
| B-809 | atrazine + nicosulfuron | isoxadifen |
| B-810 | atrazine + tembotrione | isoxadifen |
| B-811 | atrazine + topramezone | isoxadifen |
| B-812 | clomazone + glyphosate | isoxadifen |
| B-813 | diflufenican + clodinafop-propargyl | isoxadifen |
| B-814 | diflufenican + fenoxaprop-P-ethyl | isoxadifen |
| B-815 | diflufenican + flupyrsulfuron-methyl-sodium | isoxadifen |
| B-816 | diflufenican + glyphosate | isoxadifen |
| B-817 | diflufenican + mesosulfuron-methyl | isoxadifen |
| B-818 | diflufenican + pinoxaden | isoxadifen |
| B-819 | diflufenican + pyroxsulam | isoxadifen |
| B-820 | flumetsulam + glyphosate | isoxadifen |
| B-821 | flumioxazin + glyphosate | isoxadifen |
| B-822 | imazapic + glyphosate | isoxadifen |
| B-823 | imazethapyr + glyphosate | isoxadifen |
| B-824 | isoxaflutol + B-1 | isoxadifen |
| B-825 | isoxaflutol + glyphosate | isoxadifen |
| B-826 | metazachlor + B-1 | isoxadifen |
| B-827 | metazachlor + glyphosate | isoxadifen |
| B-828 | metazachlor + mesotrione | isoxadifen |
| B-829 | metazachlor + nicosulfuron | isoxadifen |
| B-830 | metazachlor + terbuthylazin | isoxadifen |
| B-831 | metazachlor + topramezone | isoxadifen |
| B-832 | metribuzin + glyphosate | isoxadifen |
| B-833 | pendimethalin + B-1 | isoxadifen |
| B-834 | pendimethalin + clodinafop-propargyl | isoxadifen |
| B-835 | pendimethalin + fenoxaprop-P-ethyl | isoxadifen |
| B-836 | pendimethalin + flupyrsulfuron-methyl-sodium | isoxadifen |
| B-837 | pendimethalin + glyphosate | isoxadifen |
| B-838 | pendimethalin + mesosulfuron-methyl | isoxadifen |
| B-839 | pendimethalin + mesotrione | isoxadifen |
| B-840 | pendimethalin + nicosulfuron | isoxadifen |
| B-841 | pendimethalin + pinoxaden | isoxadifen |
| B-842 | pendimethalin + pyroxsulam | isoxadifen |
| B-843 | pendimethalin + tembotrione | isoxadifen |
| B-844 | pendimethalin + topramezone | isoxadifen |
| B-845 | pyroxasulfone + tembotrione | isoxadifen |
| B-846 | pyroxasulfone + topramezone | isoxadifen |
| B-847 | sulfentrazone + glyphosate | isoxadifen |
| B-848 | terbuthylazin + B-1 | isoxadifen |
| B-849 | terbuthylazin + foramsulfuron | isoxadifen |
| B-850 | terbuthylazin + glyphosate | isoxadifen |
| B-851 | terbuthylazin + mesotrione | isoxadifen |
| B-852 | terbuthylazin + nicosulfuron | isoxadifen |
| B-853 | terbuthylazin + tembotrione | isoxadifen |
| B-854 | terbuthylazin + topramezone | isoxadifen |
| B-855 | trifluralin + glyphosate | isoxadifen |
| B-856 | clodinafop-propargyl | mefenpyr |
| B-857 | cycloxydim | mefenpyr |
| B-858 | cyhalofop-butyl | mefenpyr |
| B-859 | fenoxaprop-P-ethyl | mefenpyr |
| B-860 | pinoxaden | mefenpyr |
| B-861 | profoxydim | mefenpyr |
| B-862 | tepraloxydim | mefenpyr |
| B-863 | tralkoxydim | mefenpyr |
| B-864 | esprocarb | mefenpyr |
| B-865 | prosulfocarb | mefenpyr |
| B-866 | thiobencarb | mefenpyr |
| B-867 | triallate | mefenpyr |
| B-868 | bensulfuron-methyl | mefenpyr |
| B-869 | bispyribac-sodium | mefenpyr |
| B-870 | cyclosulfamuron | mefenpyr |
| B-871 | flumetsulam | mefenpyr |
| B-872 | flupyrsulfuron-methyl-sodium | mefenpyr |
| B-873 | foramsulfuron | mefenpyr |
| B-874 | imazamox | mefenpyr |
| B-875 | imazapic | mefenpyr |
| B-876 | imazapyr | mefenpyr |
| B-877 | imazaquin | mefenpyr |
| B-878 | imazethapyr | mefenpyr |
| B-879 | imazosulfuron | mefenpyr |
| B-880 | iodosulfuron-methyl-sodium | mefenpyr |
| B-881 | mesosulfuron | mefenpyr |
| B-882 | nicosulfuron | mefenpyr |
| B-883 | penoxsulam | mefenpyr |
| B-884 | propoxycarbazone-sodium | mefenpyr |
| B-885 | pyrazosulfuron-ethyl | mefenpyr |
| B-886 | pyroxsulam | mefenpyr |
| B-887 | rimsulfuron | mefenpyr |
| B-888 | sulfosulfuron | mefenpyr |
| B-889 | thiencarbazone-methyl | mefenpyr |
| B-890 | tritosulfuron | mefenpyr |
| B-891 | 2,4-D and its salts and esters | mefenpyr |
| B-892 | aminopyralid and its salts and esters | mefenpyr |
| B-893 | clopyralid and its salts and esters | mefenpyr |
| B-894 | dicamba and its salts and esters | mefenpyr |
| B-895 | fluroxypyr-meptyl | mefenpyr |
| B-896 | quinclorac | mefenpyr |
| B-897 | quinmerac | mefenpyr |
| B-898 | B-9 | mefenpyr |
| B-899 | diflufenzopyr | mefenpyr |
| B-900 | diflufenzopyr-sodium | mefenpyr |
| B-901 | clomazone | mefenpyr |
| B-902 | diflufenican | mefenpyr |
| B-903 | fluorochloridone | mefenpyr |
| B-904 | isoxaflutol | mefenpyr |
| B-905 | mesotrione | mefenpyr |
| B-906 | picolinafen | mefenpyr |
| B-907 | sulcotrione | mefenpyr |
| B-908 | tefuryltrione | mefenpyr |
| B-909 | tembotrione | mefenpyr |
| B-910 | topramezone | mefenpyr |
| B-911 | B-7 | mefenpyr |
| B-912 | atrazine | mefenpyr |
| B-913 | diuron | mefenpyr |
| B-914 | fluometuron | mefenpyr |
| B-915 | hexazinone | mefenpyr |
| B-916 | isoproturon | mefenpyr |
| B-917 | metribuzin | mefenpyr |
| B-918 | propanil | mefenpyr |
| B-919 | terbuthylazin | mefenpyr |
| B-920 | paraquat dichloride | mefenpyr |
| B-921 | flumioxazin | mefenpyr |
| B-922 | oxyfluorfen | mefenpyr |
| B-923 | sulfentrazone | mefenpyr |
| B-924 | B-1 | mefenpyr |
| B-925 | B-2 | mefenpyr |
| B-926 | glyphosate | mefenpyr |
| B-927 | glyphosate-isopropylammonium | mefenpyr |
| B-928 | glyphosate-trimesium (sulfosate) | mefenpyr |
| B-929 | glufosinate | mefenpyr |
| B-930 | glufosinate-ammonium | mefenpyr |
| B-931 | pendimethalin | mefenpyr |
| B-932 | trifluralin | mefenpyr |
| B-933 | acetochlor | mefenpyr |
| B-934 | cafenstrole | mefenpyr |
| B-935 | dimethenamid-p | mefenpyr |
| B-936 | fentrazamide | mefenpyr |
| B-937 | flufenacet | mefenpyr |
| B-938 | mefenacet | mefenpyr |
| B-939 | metazachlor | mefenpyr |
| B-940 | metolachlor-S | mefenpyr |
| B-941 | pyroxasulfone | mefenpyr |
| B-942 | isoxaben | mefenpyr |
| B-943 | dymron | mefenpyr |
| B-944 | indanofan | mefenpyr |
| B-945 | oxaziclomefone | mefenpyr |
| B-946 | triaziflam | mefenpyr |
| B-947 | atrazine + B-1 | mefenpyr |
| B-948 | atrazine + glyphosate | mefenpyr |
| B-949 | atrazine + mesotrione | mefenpyr |
| B-950 | atrazine + nicosulfuron | mefenpyr |
| B-951 | atrazine + tembotrione | mefenpyr |
| B-952 | atrazine + topramezone | mefenpyr |
| B-953 | clomazone + glyphosate | mefenpyr |
| B-954 | diflufenican + clodinafop-propargyl | mefenpyr |
| B-955 | diflufenican + fenoxaprop-P-ethyl | mefenpyr |
| B-956 | diflufenican + flupyrsulfuron-methyl-sodium | mefenpyr |
| B-957 | diflufenican + glyphosate | mefenpyr |
| B-958 | diflufenican + mesosulfuron-methyl | mefenpyr |
| B-959 | diflufenican + pinoxaden | mefenpyr |
| B-960 | diflufenican + pyroxsulam | mefenpyr |
| B-961 | flumetsulam + glyphosate | mefenpyr |
| B-962 | flumioxazin + glyphosate | mefenpyr |
| B-963 | imazapic + glyphosate | mefenpyr |
| B-964 | imazethapyr + glyphosate | mefenpyr |
| B-965 | isoxaflutol + B-1 | mefenpyr |
| B-966 | isoxaflutol + glyphosate | mefenpyr |
| B-967 | metazachlor + B-1 | mefenpyr |
| B-968 | metazachlor + glyphosate | mefenpyr |
| B-969 | metazachlor + mesotrione | mefenpyr |
| B-970 | metazachlor + nicosulfuron | mefenpyr |
| B-971 | metazachlor + terbuthylazin | mefenpyr |
| B-972 | metazachlor + topramezone | mefenpyr |
| B-973 | metribuzin + glyphosate | mefenpyr |
| B-974 | pendimethalin + B-1 | mefenpyr |
| B-975 | pendimethalin + clodinafop-propargyl | mefenpyr |
| B-976 | pendimethalin + fenoxaprop-P-ethyl | mefenpyr |
| B-977 | pendimethalin + flupyrsulfuron-methyl-sodium | mefenpyr |
| B-978 | pendimethalin + glyphosate | mefenpyr |
| B-979 | pendimethalin + mesosulfuron-methyl | mefenpyr |
| B-980 | pendimethalin + mesotrione | mefenpyr |
| B-981 | pendimethalin + nicosulfuron | mefenpyr |
| B-982 | pendimethalin + pinoxaden | mefenpyr |
| B-983 | pendimethalin + pyroxsulam | mefenpyr |
| B-984 | pendimethalin + tembotrione | mefenpyr |
| B-985 | pendimethalin + topramezone | mefenpyr |
| B-986 | pyroxasulfone + tembotrione | mefenpyr |
| B-987 | pyroxasulfone + topramezone | mefenpyr |
| B-988 | sulfentrazone + glyphosate | mefenpyr |
| B-989 | terbuthylazin + B-1 | mefenpyr |
| B-990 | terbuthylazin + foramsulfuron | mefenpyr |
| B-991 | terbuthylazin + glyphosate | mefenpyr |
| B-992 | terbuthylazin + mesotrione | mefenpyr |
| B-993 | terbuthylazin + nicosulfuron | mefenpyr |
| B-994 | terbuthylazin + tembotrione | mefenpyr |
| B-995 | terbuthylazin + topramezone | mefenpyr |
| B-996 | trifluralin + glyphosate | mefenpyr |
| B-997 | clodinafop-propargyl | B-12 |
| B-998 | cycloxydim | B-12 |
| B-999 | cyhalofop-butyl | B-12 |
| B-1000 | fenoxaprop-P-ethyl | B-12 |
| B-1001 | pinoxaden | B-12 |
| B-1002 | profoxydim | B-12 |
| B-1003 | tepraloxydim | B-12 |
| B-1004 | tralkoxydim | B-12 |
| B-1005 | esprocarb | B-12 |
| B-1006 | prosulfocarb | B-12 |
| B-1007 | thiobencarb | B-12 |
| B-1008 | triallate | B-12 |
| B-1009 | bensulfuron-methyl | B-12 |
| B-1010 | bispyribac-sodium | B-12 |
| B-1011 | cyclosulfamuron | B-12 |
| B-1012 | flumetsulam | B-12 |
| B-1013 | flupyrsulfuron-methyl-sodium | B-12 |
| B-1014 | foramsulfuron | B-12 |
| B-1015 | imazamox | B-12 |
| B-1016 | imazapic | B-12 |
| B-1017 | imazapyr | B-12 |
| B-1018 | imazaquin | B-12 |
| B-1019 | imazethapyr | B-12 |
| B-1020 | imazosulfuron | B-12 |
| B-1021 | iodosulfuron-methyl-sodium | B-12 |
| B-1022 | mesosulfuron | B-12 |
| B-1023 | nicosulfuron | B-12 |
| B-1024 | penoxsulam | B-12 |
| B-1025 | propoxycarbazone-sodium | B-12 |
| B-1026 | pyrazosulfuron-ethyl | B-12 |
| B-1027 | pyroxsulam | B-12 |
| B-1028 | rimsulfuron | B-12 |
| B-1029 | sulfosulfuron | B-12 |
| B-1030 | thiencarbazone-methyl | B-12 |
| B-1031 | tritosulfuron | B-12 |
| B-1032 | 2,4-D and its salts and esters | B-12 |
| B-1033 | aminopyralid and its salts and esters | B-12 |
| B-1034 | clopyralid and its salts and esters | B-12 |
| B-1035 | dicamba and its salts and esters | B-12 |
| B-1036 | fluroxypyr-meptyl | B-12 |
| B-1037 | quinclorac | B-12 |
| B-1038 | quinmerac | B-12 |
| B-1039 | B-9 | B-12 |
| B-1040 | diflufenzopyr | B-12 |
| B-1041 | diflufenzopyr-sodium | B-12 |
| B-1042 | clomazone | B-12 |
| B-1043 | diflufenican | B-12 |
| B-1044 | fluorochloridone | B-12 |
| B-1045 | isoxaflutol | B-12 |
| B-1046 | mesotrione | B-12 |
| B-1047 | picolinafen | B-12 |
| B-1048 | sulcotrione | B-12 |
| B-1049 | tefuryltrione | B-12 |
| B-1050 | tembotrione | B-12 |
| B-1051 | topramezone | B-12 |
| B-1052 | B-7 | B-12 |
| B-1053 | atrazine | B-12 |
| B-1054 | diuron | B-12 |
| B-1055 | fluometuron | B-12 |
| B-1056 | hexazinone | B-12 |
| B-1057 | isoproturon | B-12 |
| B-1058 | metribuzin | B-12 |
| B-1059 | propanil | B-12 |
| B-1060 | terbuthylazin | B-12 |
| B-1061 | paraquat dichloride | B-12 |
| B-1062 | flumioxazin | B-12 |
| B-1063 | oxyfluorfen | B-12 |
| B-1064 | sulfentrazone | B-12 |
| B-1065 | B-1 | B-12 |
| B-1066 | B-2 | B-12 |
| B-1067 | glyphosate | B-12 |
| B-1068 | glyphosate-isopropylammonium | B-12 |
| B-1069 | glyphosate-trimesium (sulfosate) | B-12 |
| B-1070 | glufosinate | B-12 |
| B-1071 | glufosinate-ammonium | B-12 |
| B-1072 | pendimethalin | B-12 |
| B-1073 | trifluralin | B-12 |
| B-1074 | acetochlor | B-12 |
| B-1075 | cafenstrole | B-12 |
| B-1076 | dimethenamid-P | B-12 |
| B-1077 | fentrazamide | B-12 |
| B-1078 | flufenacet | B-12 |
| B-1079 | mefenacet | B-12 |
| B-1080 | metazachlor | B-12 |
| B-1081 | metolachlor-S | B-12 |
| B-1082 | pyroxasulfone | B-12 |
| B-1083 | isoxaben | B-12 |
| B-1084 | dymron | B-12 |
| B-1085 | indanofan | B-12 |
| B-1086 | oxaziclomefone | B-12 |
| B-1087 | triaziflam | B-12 |
| B-1088 | atrazine + B-1 | B-12 |
| B-1089 | atrazine + glyphosate | B-12 |
| B-1090 | atrazine + mesotrione | B-12 |
| B-1091 | atrazine + nicosulfuron | B-12 |
| B-1092 | atrazine + tembotrione | B-12 |
| B-1093 | atrazine + topramezone | B-12 |
| B-1094 | clomazone + glyphosate | B-12 |
| B-1095 | diflufenican + clodinafop-propargyl | B-12 |
| B-1096 | diflufenican + fenoxaprop-P-ethyl | B-12 |
| B-1097 | diflufenican + flupyrsulfuron-methyl-sodium | B-12 |
| B-1098 | diflufenican + glyphosate | B-12 |
| B-1099 | diflufenican + mesosulfuron-methyl | B-12 |
| B-1100 | diflufenican + pinoxaden | B-12 |
| B-1101 | diflufenican + pyroxsulam | B-12 |
| B-1102 | flumetsulam + glyphosate | B-12 |
| B-1103 | flumioxazin + glyphosate | B-12 |
| B-1104 | imazapic + glyphosate | B-12 |
| B-1105 | imazethapyr + glyphosate | B-12 |
| B-1106 | isoxaflutol + B-1 | B-12 |
| B-1107 | isoxaflutol + glyphosate | B-12 |
| B-1108 | metazachlor + B-1 | B-12 |
| B-1109 | metazachlor + glyphosate | B-12 |
| B-1110 | metazachlor + mesotrione | B-12 |
| B-1111 | metazachlor + nicosulfuron | B-12 |
| B-1112 | metazachlor + terbuthylazin | B-12 |
| B-1113 | metazachlor + topramezone | B-12 |
| B-1114 | metribuzin + glyphosate | B-12 |
| B-1115 | pendimethalin + B-1 | B-12 |
| B-1116 | pendimethalin + clodinafop-propargyl | B-12 |
| B-1117 | pendimethalin + fenoxaprop-P-ethyl | B-12 |
| B-1118 | pendimethalin + flupyrsulfuron-methyl-sodium | B-12 |
| B-1119 | pendimethalin + glyphosate | B-12 |
| B-1120 | pendimethalin + mesosulfuron-methyl | B-12 |
| B-1121 | pendimethalin + mesotrionee | B-12 |
| B-1122 | pendimethalin + nicosulfuron | B-12 |
| B-1123 | pendimethalin + pinoxaden | B-12 |
| B-1124 | pendimethalin + pyroxsulam | B-12 |
| B-1125 | pendimethalin + tembotrione | B-12 |
| B-1126 | pendimethalin + topramezone | B-12 |
| B-1127 | pyroxasulfone + tembotrione | B-12 |
| B-1128 | pyroxasulfone + topramezone | B-12 |
| B-1129 | sulfentrazone + glyphosate | B-12 |
| B-1130 | terbuthylazin + B-1 | B-12 |
| B-1131 | terbuthylazin + foramsulfuron | B-12 |
| B-1132 | terbuthylazin + glyphosate | B-12 |
| B-1133 | terbuthylazin + mesotrione | B-12 |
| B-1134 | terbuthylazin + nicosulfuron | B-12 |
| B-1135 | terbuthylazin + tembotrione | B-12 |
| B-1136 | terbuthylazin + topramezone | B-12 |
| B-1137 | trifluralin + glyphosate | B-12 |
| B-1138 | 2-1 | — |
| B-1139 | 2-2 | — |
| B-1140 | 2-3 | — |
| B-1141 | 2-4 | — |
| B-1142 | 2-5 | — |
| B-1143 | 2-6 | — |
| B-1144 | 2-7 | — |
| B-1145 | 2-8 | — |
| B-1146 | 2-9 | — |
| B-1147 | 2-10 | — |
| B-1148 | 2-1 | benoxacor |
| B-1149 | 2-2 | benoxacor |
| B-1150 | 2-3 | benoxacor |
| B-1151 | 2-4 | benoxacor |
| B-1152 | 2-5 | benoxacor |
| B-1153 | 2-6 | benoxacor |
| B-1154 | 2-7 | benoxacor |
| B-1155 | 2-8 | benoxacor |
| B-1156 | 2-9 | benoxacor |
| B-1157 | 2-10 | benoxacor |
| B-1158 | 2-1 | cloquintocet |
| B-1159 | 2-2 | cloquintocet |
| B-1160 | 2-3 | cloquintocet |
| B-1161 | 2-4 | cloquintocet |
| B-1162 | 2-5 | cloquintocet |
| B-1163 | 2-6 | cloquintocet |
| B-1164 | 2-7 | cloquintocet |
| B-1165 | 2-8 | cloquintocet |
| B-1166 | 2-9 | cloquintocet |
| B-1167 | 2-10 | cloquintocet |
| B-1168 | 2-1 | cyprosulfamide |
| B-1169 | 2-2 | cyprosulfamide |
| B-1170 | 2-3 | cyprosulfamide |
| B-1171 | 2-4 | cyprosulfamide |
| B-1172 | 2-5 | cyprosulfamide |
| B-1173 | 2-6 | cyprosulfamide |
| B-1174 | 2-7 | cyprosulfamide |
| B-1175 | 2-8 | cyprosulfamide |
| B-1176 | 2-9 | cyprosulfamide |
| B-1177 | 2-10 | cyprosulfamide |
| B-1178 | 2-1 | dichlormid |
| B-1179 | 2-2 | dichlormid |
| B-1180 | 2-3 | dichlormid |
| B-1181 | 2-4 | dichlormid |
| B-1182 | 2-5 | dichlormid |
| B-1183 | 2-6 | dichlormid |
| B-1184 | 2-7 | dichlormid |
| B-1185 | 2-8 | dichlormid |
| B-1186 | 2-9 | dichlormid |
| B-1187 | 2-10 | dichlormid |
| B-1188 | 2-1 | fenchlorazole |
| B-1189 | 2-2 | fenchlorazole |
| B-1190 | 2-3 | fenchlorazole |
| B-1191 | 2-4 | fenchlorazole |
| B-1192 | 2-5 | fenchlorazole |
| B-1193 | 2-6 | fenchlorazole |
| B-1194 | 2-7 | fenchlorazole |
| B-1195 | 2-8 | fenchlorazole |
| B-1196 | 2-9 | fenchlorazole |
| B-1197 | 2-10 | fenchlorazole |
| B-1198 | 2-1 | isoxadifen |
| B-1199 | 2-2 | isoxadifen |
| B-1200 | 2-3 | isoxadifen |
| B-1201 | 2-4 | isoxadifen |
| B-1202 | 2-5 | isoxadifen |
| B-1203 | 2-6 | isoxadifen |
| B-1204 | 2-7 | isoxadifen |
| B-1205 | 2-8 | isoxadifen |
| B-1206 | 2-9 | isoxadifen |
| B-1207 | 2-10 | isoxadifen |
| B-1208 | 2-1 | mefenpyr |
| B-1209 | 2-2 | mefenpyr |
| B-1210 | 2-3 | mefenpyr |
| B-1211 | 2-4 | mefenpyr |
| B-1212 | 2-5 | mefenpyr |
| B-1213 | 2-6 | mefenpyr |
| B-1214 | 2-7 | mefenpyr |
| B-1215 | 2-8 | mefenpyr |
| B-1216 | 2-9 | mefenpyr |
| B-1217 | 2-10 | mefenpyr |
| B-1218 | 2-1 | B-11 |
| B-1219 | 2-2 | B-11 |
| B-1220 | 2-3 | B-11 |
| B-1221 | 2-4 | B-11 |
| B-1222 | 2-5 | B-11 |
| B-1223 | 2-6 | B-11 |
| B-1224 | 2-7 | B-11 |
| B-1225 | 2-8 | B-11 |
| B-1226 | 2-9 | B-11 |
| B-1227 | 2-10 | B-11 |
| B-1228 | 2-1 | B-12 |
| B-1229 | 2-2 | B-12 |
| B-1230 | 2-3 | B-12 |
| B-1231 | 2-4 | B-12 |
| B-1232 | 2-5 | B-12 |
| B-1233 | 2-6 | B-12 |
| B-1234 | 2-7 | B-12 |
| B-1235 | 2-8 | B-12 |
| B-1236 | 2-9 | B-12 |
| B-1237 | 2-10 | B-12 |

The compounds I and the compositions according to theinvention may also have a plant-strengthening action. Accordingly, they are suitable for mobilizing the defense system of the plants against attack by unwanted microorganisms, such as harmful fungi, but also viruses and bacteria. Plant-strengthening (resistance-inducing) substances are to be understood as meaning, in the present context, those substances which are capable of stimulating the defense system of treated plants in such a way that, when subsequently inoculated by unwanted microorganisms, the treated plants display a substantial degree of resistance to these microorganisms.

The compounds I can be employed for protecting plants against attack by unwanted microorganisms within a certain period of time after the treatment. The period of time within which their protection is effected generally extends from 1 to 28 days, preferably from 1 to 14 days, after the treatment of the plants with the compounds I, or, after treatment of the seed, for up to 9 months after sowing.

The compounds I and the compositions according to the invention are also suitable for increasing the harvest yield.

Moreover, they have reduced toxicity and are tolerated well by the plants.

### SYNTHESIS EXAMPLES

With appropriate modification of the starting materials, the procedures given in the synthesis examples below were used to obtain further compounds I. The compounds thus obtained are recited in the following table with physical data:

### I. PREPARATION EXAMPLES

### Example 1

### Preparation of methyl 3-(3-hydroxypyridin-2-yl)-3-oxo-2-(2-trifluoromethylphenyl)propionate [I-1]

### Step 1: Methyl 3-(4-methoxybenzyloxy)pyridine-2-carboxylate

4 g of NaH were added to a solution of 15.2 g of methyl 3-hydroxypyridine-2-carboxylate in 200 ml of dimethyl formamide (DMF) and the mixture was stirred at 20-25° C. for 30 min. 15.4 g of para-methoxybenzyl chloride were then added. The mixture was stirred at 40° C. for about 15 hours and then added to water. After extraction with ethyl acetate, phase separation and washing of the combined organic phases with water and saturated NaCl solution followed by drying and removal of the solvent, the residue was subjected to column chromatography. This gave 20 g of the title compound as a yellow oil.

### Step 2: 3-(4-Methoxybenzyloxy)pyridine-2-carboxylic acid

A solution of 7 g of the ester from step 1 and 2.2 g of LiOH in 60 ml of methanol and 40 ml of water was stirred at 20-25° C. for about 15 hours. 100 ml of water and 7 ml of acetic acid were then added. The mixture was extracted with ethyl acetate. The combined organic phases were dried and freed from the solvent This gave 5 g of the title compound as a yellowish solid.

### Step 3: Pentafluorophenyl 3-(4-methoxybenzyloxy)pyridine-2-carboxylate

2.4 g of N,N′-diisopropylcarbodiimide were added dropwise to a solution of 5 g of the acid from step 2 and 3.5 g of pentafluorophenol in 100 nil of CH₂Cl₂. The mixture was stirred at 20-25° C. for about 1 hour, then washed with saturated NaCl solution and dried under reduced pressure. Purification of the residue by column chromatography (petroleum ether:ethyl acetate 5:1) gave 4.6 g of the title compound.

### Step 4: Methyl 3-[3-(4-methoxybenzyloxy)pyridin-2-yl]-3-oxo-2-(2-trifluoromethyl-phenyl)propionate

0.74 g of potassium tert-butoxide was added to a solution of 1.2 g of the ester from step 3 in 10 ml DMF. The mixture was stirred at 20-25° C. for about 30 min. A solution of 2.25 g of methyl (2-trifluoromethylphenyl)acetate in 5 ml of DMF was then added. After 30 min of stirring at 20-25° C., the reaction was ended by addition of saturated NH₄Cl solution. The mixture was extracted with ethyl acetate. The combined organic phases were washed with saturated NaCl solution and then dried and freed from the solvent. Purification of the residue by column chromatography (petroleum ether:ethyl acetate 5:1) gave 0.23 g of the title compound.

### Step 5: Methyl 3-(3-hydroxypyridin-2-yl)-3-oxo-2-(2-trifluoromethylphenyl)propionate

At 0° C., 1 ml of trifluoroacetic acid was added to 160 mg of the ester from step 4, and the reaction mixture was stirred at 0° C. for 20 min. Saturated NaHCO₃ solution was then added. Extraction with ethyl acetate, removal of the solvent under reduced pressure and preparative HPLC gave 83 mg of the title compound.

¹H NMR (CDCl₃): δ 11.05 (brs, 1H); 8.26-8.24 (m, 1H); 7.73 (d, 1H); 7.61-7.58 (m, 2H); 7.48-7.34 (m, 3H); 6.64 (s, 1H); 3.76 (s, 3H).

### Example 2

### Preparation of methyl 3-acetoxy-3-(3-acetoxypyridin-2-yl)-2-(2-trifluoromethylphenyl)acrylate [I-2]

A solution of methyl 3-hydroxy-3-(3-hydroxypyridin-2-yl)-2-(2-trifluoromethylphenyl)-acrylate (from Example 1) in 5 ml of toluene was admixed at 20 to 25° C. with 414 mg of acetyl chloride and 210 mg of pyridine. The mixture was stirred at 100° C. for 2 h. The solvent was removed under reduced pressure and an aqueous HCl solution was added. Extraction with ethyl acetate, washing with saturated NaCl solution, drying and removal of the solvent under reduced pressure and purification by preparative HPLC gave 90 mg of the title compound.

TABLE I
Compounds of formula I that correspond to formulae I.1A, I.2A, I.1T, and I.2T, respectively:

I.1A

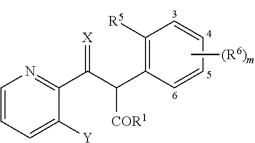

I.2A

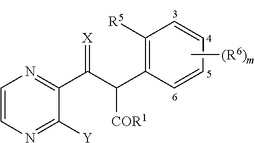

I.1T

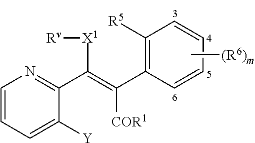

I.2T

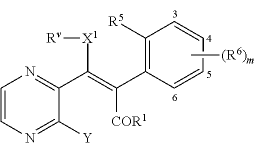

| | | | X/ | | | | | Physical data |
|---|---|---|---|---|---|---|---|---|
| No. | Formula | Y | X¹ | R^{v} | R¹ | R⁵ | (R⁶)ₘ | ¹H NMR(δ [ppm])*⁾ |
| I-1 | I.1A | OH | O | — | OCH₃ | CF₃ | — | 11.04 (s), 8.24 (d), 7.74 (d), |
| | | | | | | | | 7.59 (m), 7.49 (m), 7.42 (m), |
| | | | | | | | | 7.36 (m), 6.64 (s), 3.76 (s) |
| I-2 | I.1T | O(C═O)CH₃ | O | (C═O)CH₃ | OCH₃ | CF₃ | — | 8.52 (d), 7.72 (m), 7.69 (t), |
| | | | | | | | | 7.51 (t), 7.48 (m), 3.51 (s), |
| | | | | | | | | 2.43 (s), 1.79 (s) |
| I-3 | I.1T | O(C═O)C(CH₃)₃ | O | (C═O)CH(CH₃)₃ | OCH₃ | CF₃ | — | 8.52 (d), 7.72 (d), 7.58 (t), |
| | | | | | | | | 7.49 (t), 7.42 (d), 7.35 (s), |
| | | | | | | | | 3.51 (s), 1.39 (s), 0.84 (s) |
| I-4 | I.1T | O(C═O)CH(CH₃)₂ | O | (C═O)CH(CH₃)₂ | OCH₃ | CF₃ | — | 8.52 (d), 7.74 (d), 7.58 (m), |
| | | | | | | | | 7.44 (m), 7.33 (d), 3.50 (s), |
| | | | | | | | | 2.82 (m), 2.31 (m), 1.42 (m), |
| | | | | | | | | 0.81 (d), 0.68 (d) |
| I-5 | I.1A | OH | O | — | OCH₂ | CF₃ | — | 11.06 (s), 8.25 (d), 7.72 (d), |
| | | | | | CH₃ | | | 7.61 (t), 7.48 (m), 7.42 (t), |
| | | | | | | | | 7.35 (d), 6.62 (s), 4.24 (m), |
| | | | | | | | | 1.22 (t) |
| I-6 | I.1A | OH | O | — | OCH₃ | CHF₂ | — | 8.24 (d), 7.62 (m), 7.52 (m), |
| | | | | | | | | 7.44 (m), 7.36 (m), 7.14 (t), |
| | | | | | | | | 6.66 (s), 3.78 (s) |
| I-7 | I.1T | O(C═O)C(CH₃)₃ | O | (C═O)C(CH₃)₃ | OCH₃ | CHF₂ | — | 8.52 (d), 7.71 (d), 7.60 (d), |
| | | | | | | | | 7.50 (m), 7.39 (m), 7.35 (d), |
| | | | | | | | | 6.97-6.70 (t), 3.52 (s), |
| | | | | | | | | 1.40 (s) |
| I-8 | I.1A | OH | O | — | OCH₃ | Cl | 4- | 8.25-8.24 (m), 7.46- |
| | | | | | | | OCHF₂, | 7.43 (m), 7.37-7.28 (m), |
| | | | | | | | 6-Cl | 7.26 (s), 7.16 (s), 3.76 (s) |
| I-9 | I.1A | OH | O | — | OCH₃ | Cl | 4-CF₃, | 10.95 (s), 8.24- |
| | | | | | | | 6-Cl | 8.22 (m), 7.64 (s), 7.43- |
| | | | | | | | | 7.37 (m), 7.21 (s), 3.77 (s). |
| I-10 | I.1A | OH | O | — | OCH₃ | OCF₃ | — | 11.10 (s) 8.25 (d), 7.54 (d), |
| | | | | | | | | 7.45-7.30 (m), 6.64 (s), |
| | | | | | | | | 3.76 (s) |
| I-11 | I.1T | OCH₂CF₃ | O | CH₂CF₃ | OCH₃ | CF₃ | — | 8.50 (d), 7.72 (d), 7.59 (m), |
| | | | | | | | | 7.52-5.41 (m), 4.47 (m), |
| | | | | | | | | 3.86 (m), 3.45 (s) |
| I-12 | I.1T | OCH₂CHF₂ | O | CH₂CHF₂ | OCH₃ | CF₃ | — | 8.32 (d), 7.63 (d), 7.50 (m), |
| | | | | | | | | 7.34 (m), 7.24 (d), 6.16- |
| | | | | | | | | 5.88 (t), 5.65-5.41 (t), |
| | | | | | | | | 4.17 (m), 3.59 (t), 3.33 (s) |
| I-13 | I.1T | O(C═O)CH₃ | O | (C═O)CH₃ | OCH₃ | OCF₃ | — | 8.53 (d), 7.60 (d), 7.35 (m), |
| | | | | | | | | 3.56 (s), 2.29 (s), 1.93 (s) |
| I-14 | I.1A | NH(C═O)C(CH₃)₃ | O | — | OCH₃ | CF₃ | — | 11.39 (s), 9.17 (d), 8.34 (s), |
| | | | | | | | | 7.73 (d), 7.60 (t), 7.54 (d), |
| | | | | | | | | 7.49-7.45 (m), 6.69 (s), |
| | | | | | | | | 3.75 (s), 1.32 (s) |
| I-15 | I.1A | NHCH₂CHF₂ | O | — | OCH₃ | CF₃ | — | 8.61 (t), 8.02 (d), 7.72 (d), |
| | | | | | | | | 7.59 (d), 7.45 (m), 7.31 (m), |
| | | | | | | | | 7.18 (d), 6.63 (s), 6.02- |
| | | | | | | | | 5.73 (t), 3.74 (s), 3.61 (t) |
| I-16 | I.1A | SH | O | — | OCH₃ | CF₃ | — | 8.43 (d), 7.70 (d), 7.63- |
| | | | | | | | | 7.53 (m), 7.43 (t), 7.26- |
| | | | | | | | | 7.23 (m), 6.59 (s), 4.76 (s), |
| | | | | | | | | 3.74 (s) |
| I-17 | I.2A | OH | O | — | OCH₃ | CF₃ | — | 11.81 (s), 8.27 (s), 8.18 (s), |
| | | | | | | | | 7.78 (d), 7.65-7.31 (m), |
| | | | | | | | | 6.62 (s), 3.81 (s) |
| I-18 | I.2T | O(C═O)C(CH₃)₃ | O | (C═O)C(CH₃)₃ | OCH₃ | CF₃ | — | 8.53 (d), 8.40 (d), 7.73 (d), |
| | | | | | | | | 7.57 (t), 7.51 (t), 7.39 (d), |
| | | | | | | | | 3.55 (s), 1.41 (s), 0.84 (s) |

### USE EXAMPLES

The herbicidal activity of the compounds of the formula I was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active compounds, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this has been impaired by the active compounds.

For the post-emergence treatment, the test plants were first grown to a height of 3 to 15 cm, depending on the plant habit, and then treated with the active compounds which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

The plants used in the greenhouse experiments belonged to the following species:

| Bayer code | Scientific name | Common name |
|---|---|---|
| ABUTH | Abutilon theophrasti | China jute |
| ALOMY | Alopecurus myosuroides | Blackgrass |
| AMARE | Amaranthus retroflexus | Carelessweed |
| APESV | Apera spica-venti | Corngrass |
| AVEFA | Avena fatua | spring wild-oat |
| CHEAL | Chenopodium album | Pigweed |
| GALAP | Galium aparine | Goosegrass |
| SETFA | Setaria faberi | giant foxtail |
| SETIT | Setaria italica | foxtail millet |

1) At an application rate of 3.0 kg/ha, the active compound I-1, applied by the pre-emergence method, showed good herbicidal activity against ABUTH and very good herbicidal activity against SETIT.

2) At an application rate of 3.0 kg/ha, the active compounds I-1, I-8 and I-9, applied by the post-emergence method, showed very good herbicidal activity against ABUTH.

3) Applied by the post-emergence method at an application rate of 3.0 kg/ha, the active compound I-1 showed good and the active compound I-9 very good herbicidal activity against ALOMY.

4) Very good herbicidal activity against AMARE was shown in post-emergence by active compound I-1 at an application rate of 0.25 kg/ha and active compounds I-2, I-3, I-4, I-6* and I-7 at 0.5 kg/ha and active compound I-5 at 1.0 kg/ha.

5) Active compound I-17 showed very good herbicidal activity against APESV in post-emergence at an application rate of 1.0 kg/ha.

6) The active compounds I-1 and I-8 showed very good herbicidal activity against AVEFA in post-emergence at an application rate of 3.0 kg/ha.

7) Very good herbicidal activity against CHEAL was shown in post-emergence by active compound I-1 at an application rate of 0.25 kg/ha, by active compounds I-2, I-3, I-4, I-6* and I-7 at 0.5 kg/ha and by active compound I-5 at 1.0 kg/ha.

8) Active compound I-17 showed very good herbicidal activity against CHEAL in post-emergence at an application rate of 1.0 kg/ha.

9) Active compound I-1 showed very good herbicidal activity against GALAP in post-emergence at an application rate of 0.25 kg/ha.

10) Active compound I-1 showed very good herbicidal activity against SETFA in post-emergence at an application rate of 3.0 kg/ha.

* application rate 0.528 kg/ha

1-15. (canceled)

16. A compound of the formula I 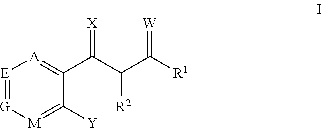
wherein:
R¹ is OR, SR or NRⁱRⁱⁱ;
R is hydrogen, C₁-C₆-alkyl, C₁-C₄-haloalkyl, C₂-C₆-alkenyl, C₃-C₆-alkynyl, Z—C₃-C₁₀-cycloalkyl, C₁-C₆-alkoxy-C₁-C₆-alkyl, C₁-C₆-cyanoalkyl, Z-phenyl, Z—C(═O)—R^{a2}, Z—SO₂R^{a2} or tri-C₁-C₄-alkylsilyl;
R^{a2} is C₁-C₆-alkyl, C₁-C₄-haloalkyl, Z—C₁-C₆-alkoxy, Z—C₁-C₄-haloalkoxy or NRⁱRⁱⁱ;
Z is a covalent bond or C₁-C₄-alkylene;
Rⁱ, Rⁱⁱ independently of one another are hydrogen, C₁-C₈-alkyl, C₁-C₄-haloalkyl, C₃-C₈-alkenyl, C₃-C₈-alkynyl, Z—C₃-C₆-cycloalkyl, Z—(C═O)—R^{a2}, Z—C₁-C₈-alkoxy, or Z—C₁-C₈-haloalkoxy; or
Rⁱ and Rⁱⁱ together with the nitrogen atom to which they are attached may form a 5- or 6-membered monocyclic or 9- or 10-membered bicyclic heterocycle which comprises 1, 2, 3 or 4 heteroatoms selected from the group consisting of O, N and S;
R² is phenyl, naphthyl or a 5- or 6-membered monocyclic or 9- or 10-membered bicyclic aromatic heterocycle which comprises 1, 2, 3 or 4 heteroatoms selected from the group consisting of O, N and S, where the cyclic groups are unsubstituted or substituted by 1, 2, 3, 4 or 5 groups R^{a};
R^{a} independently of one another are Z—CN, Z—OH, Z—NO₂, Z-halogen, C₁-C₈-alkyl, C₁-C₄-haloalkyl, C₂-C₈-alkenyl, C₂-C₈-alkynyl, Z—C₁-C₈-alkoxy, Z—C₁-C₈-halo-alkoxy, Z—C₃-C₁₀-cycloalkyl, O—Z—C₃-C₁₀-cycloalkyl, Z—C(═O)—R^{a2}, NRⁱRⁱⁱ, Z-(tri-C₁-C₄-alkyl)silyl, Z-phenyl or S(O)ₙR^{bb},
wherein R^{bb} is C₁-C₈-alkyl, C₁-C₆-haloalkyl or 2-phenyl and
n is 0, 1 or 2; or
R^{a} together with the group R^{a} attached to the adjacent carbon atom may form a five- or six-membered saturated or partially or fully unsaturated ring which, in addition to carbon atoms, may comprise 1, 2 or 3 heteroatoms selected from the group consisting of O, N and S;
X is O, S or N—R;
Y is R¹ or NH—R;
W is O or S;
A, E, G, M are N or C—R^{c}, one or two of these groups being N;
R^{c} is hydrogen or one of the groups mentioned for R^{a};
where in the groups R¹, R², R³ and their substituents, the carbon chains and/or the cyclic groups may be partially or fully substituted by groups R^{a},
or a N-oxide or an agriculturally suitable salt thereof.

17. The compound of claim 16 which corresponds to the formula I′ 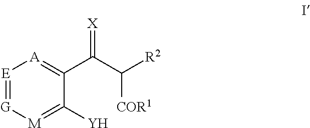
wherein:
R¹ is OR or NRⁱRⁱⁱ;
R is hydrogen, C₁-C₆-alkyl, C₁-C₄-haloalkyl, C₂-C₆-alkenyl, C₃-C₆-alkynyl, Z—C₃-C₁₀-cycloalkyl, C₁-C₆-alkoxy-C₁-C₆-alkyl, C₁-C₆-cyanoalkyl, Z-phenyl, Z—C(═O)—R^{a2} or tri-C₁-C₄-alkylsilyl;
R^{a2} is C₁-C₆-alkyl, C₁-C₄-haloalkyl, Z—C₁-C₆-alkoxy, Z—C₁-C₄-haloalkoxy or NRⁱRⁱⁱ;
Z is a covalent bond or C₁-C₄-alkylene;
Rⁱ, Rⁱⁱ independently of one another are hydrogen, C₁-C₈-alkyl, C₁-C₄-haloalkyl, C₃-C₈-alkenyl, C₃-C₈-alkynyl, Z—C₃-C₆-cycloalkyl, Z—C₁-C₈-alkoxy, or Z—C₁-C₈-haloalkoxy;
or
Rⁱ and Rⁱⁱ together with the nitrogen atom to which they are attached may form a 5- or 6-membered monocyclic or 9- or 10-membered bicyclic heterocycle which comprises 1, 2, 3 or 4 heteroatoms selected from the group consisting of O, N and S;
R² is phenyl or naphthyl, wherein the cyclic groups are unsubstituted or substituted by 1, 2, 3
or 4 groups R^{a};
R^{a} independently of one another are Z—CN, Z—OH, Z—NO₂, Z-halogen, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, Z—C₃-C₁₀-cycloalkyl, O—Z—C₃-C₁₀-cycloalkyl, Z—C(═O)—R^{a2}, NRⁱRⁱⁱ, Z-(tri-C₁-C₄-alkyl)silyl, Z-phenyl or S(O)ₙR^{bb},
where R^{bb} is C₁-C₈-alkyl or C₁-C₆-haloalkyl and
n is 0, 1 or 2; or
R^{a} together with the group R^{a} attached to the adjacent carbon atom may form a five- or six-membered saturated or partially or fully unsaturated ring which, in addition to carbon atoms, may comprise 1, 2 or 3 heteroatoms selected from the group consisting of O, N and S;
X, Y are O, S or N—R;
A, E, G, M are N or C—R^{c}, one of these groups being N;
R^{c} is hydrogen or one of the groups mentioned for R^{a};
where in the groups R¹, R², R³ and their substituents, the carbon chains and/or the cyclic groups may be partially or fully substituted by groups R^{a}.

18. The compound of claim 1, wherein Y is OH.

19. The compound of claim 1 , wherein Y is SH.

20. The compound of claim 1, wherein Y is NH—R.

21. The compound claim 1 in which R¹ is C₁-C₄-alkoxy.

22. The compound of claim 1,which corresponds to the formula I.1 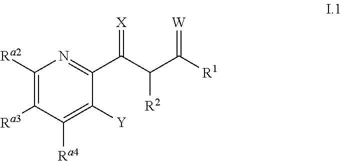
wherein R^{a2}, R^{a3} and R^{a4} each correspond to a group R^{a}.

23. The compound of claim 1, which corresponds to the formula I.A 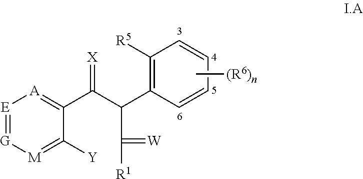
wherein R⁵ and R⁶ are groups R^{a} and n is an integer from zero to four.

24. The compound of claim 1, which corresponds to the formula I¹ 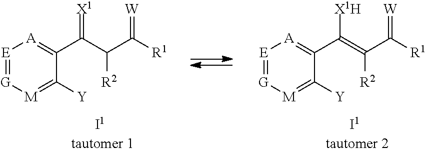
wherein X¹ is O or S.

25. The compound of claim 1, which corresponds to the formula I.T 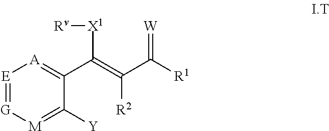
wherein R^{v} is C₁-C₈-alkyl, C₁-C₈-alkylcarbonyl, C₁-C₈-alkylsulfonyl, C₁-C₈-arylcarbonyl or C₁-C₈-arylsulfonyl where the alkyl and aryl groups may each be partially or fully substituted by groups R^{a}.

26. The compound of claim 1, wherein X is O.

27. A composition comprising a herbicidally effective amount of at least one compound of claim 1 and auxiliaries customary for formulating crop protection agents.

28. The composition according to claim 27 which comprises at least one further active compound.

29. The composition of claim 28 which comprises two further active compounds from the group of the herbicides and/or safeners.

30. A method for controlling unwanted vegetation which comprises allowing a herbicidally effective amount of at least one compound of claim 1 to act on plants, their seed and/or their habitat.

31. The method of claim 30 in which the compound corresponds to the formula I′ 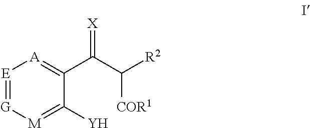
wherein:
R¹ is OR or NRⁱRⁱⁱ;
R is hydrogen, C₁-C₆-alkyl, C₁-C₄-haloalkyl, C₂-C₆-alkenyl, C₃-C₆-alkynyl, Z—C₃-C₁₀-cycloalkyl, C₁-C₆-alkoxy-C₁-C₆-alkyl, C₁-C₆-cyanoalkyl, Z-phenyl, Z—C(═O)—R^{a2} or tri-C₁-C₄-alkylsilyl;
R^{a2} is C₁-C₆-alkyl, C₁-C₄-haloalkyl, Z—C₁-C₆-alkoxy, Z—C₁-C₄-haloalkoxy or NRⁱRⁱⁱ;
Z is a covalent bond or C₁-C₄-alkylene;
Rⁱ, Rⁱⁱ independently of one another are hydrogen, C₁-C₈-alkyl, C₁-C₄-haloalkyl, C₃-C₈-alkenyl, C₃-C₈-alkynyl, Z—C₃-C₆-cycloalkyl, Z—C₁-C₈-alkoxy, or Z—C₁-C₈-haloalkoxy;
or
Rⁱ and Rⁱⁱ together with the nitrogen atom to which they are attached may form a 5- or 6-membered monocyclic or 9- or 10-membered bicyclic heterocycle which comprises 1, 2, 3 or 4 heteroatoms selected from the group consisting of O, N and S;
R² is phenyl or naphthyl, wherein the cyclic groups are unsubstituted or substituted by 1, 2, 3 or 4 groups R^{a};
R^{a} independently of one another are Z—CN, Z—OH, Z—NO₂, Z-halogen, C₁-C₈-alkyl, C₁-C₄-haloalkyl, C₂-C₈-alkenyl, C₂-C₈-alkynyl, Z—C₁-C₈-alkoxy, Z—C₁-C₈-haloalkoxy, O—Z—C₃-C₁₀-cycloalkyl, Z—C(═O)—R^{a2}, NRⁱRⁱⁱ, Z-(tri-C₁-C₄-alkyl)silyl, Z-phenyl or S(O)ₙR^{bb},
where R^{bb} is C₁-C₈-alkyl or C₁-C₆-haloalkyl and
n is 0, 1 or 2; or
R^{a} together with the group R^{a} attached to the adjacent carbon atom may form a five- or six-membered saturated or partially or fully unsaturated ring which, in addition to carbon atoms, may comprise 1, 2 or 3 heteroatoms selected from the group consisting of O, N and S;
X, Y are O, S or N—R;
A, E, G, M are N or C—R^{c}, one of these groups being N;
R^{c} is hydrogen or one of the groups mentioned for R^{a};
where in the groups R¹, R², R³ and their substituents, the carbon chains and/or the cyclic groups may be partially or fully substituted by groups R^{a}.

32. The method of claim 30, wherein Y is OH.

33. The method of claim 30, wherein Y is SH.

34. The method of claim 30, wherein Y is NH—R.

35. The method of claim 30, wherein R¹ is C₁-C₄-alkoxy.

36. The method of claim 30, in which the compound corresponds to the formula I.1 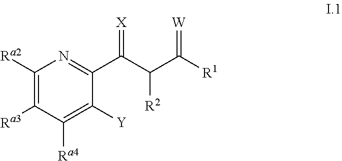
wherein R^{a2}, R^{a3} and R^{a4} each correspond to a group R^{a}.

37. The method of claim 30, in which the compound corresponds to the formula I.A 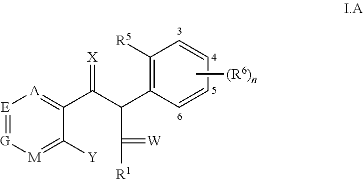
wherein R⁵ and R⁶ are groups R^{a} and n is an integer from zero to four.

38. The method of claim 30, in which the compound corresponds to the formula I¹ 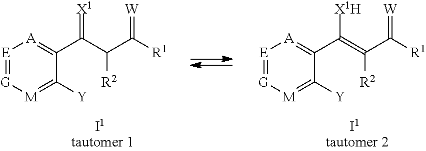
wherein X¹ is O or S.

39. The method of claim 30, in which the compound corresponds to the formula I.T 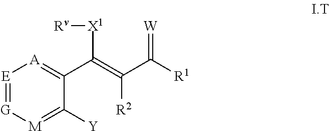
wherein R^{v} is C₁-C₈-alkyl, C₁-C₈-alkylcarbonyl, C₁-C₈-alkylsulfonyl, C₁-C₈-arylcarbonyl or C₁-C₈-arylsulfonyl where the alkyl and aryl groups may each be partially or fully substituted by groups R^{a}.

40. The method of claim 30, wherein X is O.
